(19) United States
(12) Patent Application Publication
(10) Pub. No.: US 2019/0103182 A1
(43) Pub. Date: Apr. 4, 2019

(54) MANAGEMENT OF COMFORT STATES OF AN ELECTRONIC DEVICE USER

(71) Applicant: Apple Inc., Cupertino, CA (US)

(72) Inventors: Volodymyr Borshch, Cupertino, CA (US); Mahdi Nezamabadi, San Jose, CA (US)

(21) Appl. No.: 16/019,788

(22) Filed: Jun. 27, 2018

Related U.S. Application Data
(60) Provisional application No. 62/565,390, filed on Sep. 29, 2017.

(51) Int. Cl. G16H 20/30; G06F 17/30; G06N 99/00

(52) U.S. Cl. CPC G16H 20/30; G06N 99/005; G06F 17/30528

(57) ABSTRACT

Systems, methods, and computer-readable media for managing comfort states of a user of an electronic device are provided that may train and utilize any suitable comfort model in conjunction with any suitable environment data when determining a predicted comfort state of a user at a particular environment (e.g., generally, at a particular time, and/or for performing a particular activity).

### CROSS-REFERENCE TO RELATED APPLICATION(S)

This application claims the benefit of prior filed U.S. Provisional Patent Application No. 62/565,390, filed Sep. 29, 2017, which is hereby incorporated by reference herein in its entirety.

### TECHNICAL FIELD

This disclosure relates to the management of comfort states of an electronic device user and, more particularly, to the management of comfort states of an electronic device user with a trained comfort model.

### BACKGROUND OF THE DISCLOSURE

An electronic device (e.g., a cellular telephone) may be provided with one or more sensing components (e.g., light sensors, sound sensors, location sensors, etc.) that may be utilized for attempting to determine a type of environment in which the electronic device is situated. However, the data provided by such sensing components is insufficient on its own to enable a reliable determination of a comfort state of a user of such an electronic device in a particular environment.

### SUMMARY OF THE DISCLOSURE

This document describes systems, methods, and computer-readable media for managing comfort states of a user of an electronic device.

For example, a method for managing a comfort level of an experiencing entity using a comfort model custodian system is provided, wherein the method may include initially configuring, at the comfort model custodian system, a learning engine for the experiencing entity, receiving, at the comfort model custodian system from the experiencing entity, environment category data for at least one environment category for an environment and a score for the environment, training, at the comfort model custodian system, the learning engine using the received environment category data and the received score, accessing, at the comfort model custodian system, environment category data for the at least one environment category for another environment, scoring the other environment, using the learning engine for the experiencing entity at the comfort model custodian system, with the accessed environment category data for the other environment, and when the score for the other environment satisfies a condition, generating, with the comfort model custodian system, control data associated with the satisfied condition.

As another example, a comfort model custodian system is provided that may include a communications component and a processor operative to initially configure a learning engine for an experiencing entity, receive, from the experiencing entity via the communications component, environment category data for at least one environment category for an environment and a score for the environment, train the learning engine using the received environment category data and the received score, access environment category data for the at least one environment category for another environment, score the other environment, using the learning engine for the experiencing entity, with the accessed environment category data for the other environment, and, when the score for the other environment satisfies a condition, generate control data associated with the satisfied condition.

As yet another example, a non-transitory computer-readable storage medium storing at least one program including instructions is provided, which, when executed may initially configure a learning engine for an experiencing entity, receive, from the experiencing entity, environment category data for at least one environment category for an environment and a score for the environment, train the learning engine using the received environment category data and the received score, access environment category data for the at least one environment category for another environment, score the other environment, using the learning engine for the experiencing entity at the comfort model custodian system, with the accessed environment category data for the other environment, and, when the score for the other environment satisfies a condition, generate control data associated with the satisfied condition.

This Summary is provided only to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figures, and Claims.

### BRIEF DESCRIPTION OF THE DRAWINGS

The discussion below makes reference to the following drawings, in which like reference characters refer to like parts throughout, and in which:

FIG. 1 is a schematic view of an illustrative system with an electronic device for managing comfort states;

FIG. 2 is a diagram of various illustrative environments in which the system of FIG. 1 may be used to manage comfort states;

FIG. 3 is a schematic view of an illustrative portion of the electronic device of FIGS. 1 and 2; and

FIG. 4 is a flowchart of an illustrative process for managing comfort states.

### DETAILED DESCRIPTION OF THE DISCLOSURE

Systems, methods, and computer-readable media may be provided to manage comfort states of a user of an electronic device (e.g., to determine a comfort state of an electronic device user and to manage a mode of operation of the electronic device or an associated subsystem based on the determined comfort state). Any suitable comfort model (e.g., neural network and/or learning engine) may be trained and utilized in conjunction with any suitable environment data that may be indicative of any suitable characteristics of an environment (e.g., location, temperature, humidity, white point chromaticity, illuminance, noise level, air velocity, oxygen level, harmful gas level, etc.) and/or any suitable user behavior when exposed to such an environment in order to predict or otherwise determine an appropriate comfort state of a user at a particular environment (e.g., generally, at a particular time, and/or for performing a particular activity). Such a comfort state may be analyzed with respect to particular conditions or regulations or thresholds in order to generate any suitable control data for controlling any suitable functionality of any suitable output assembly of the electronic device or of any subsystem associated with the environment (e.g., for adjusting a user interface presentation to a user (e.g., to provide a comfort suggestion or a comfort score) and/or for adjusting an output that may affect the comfort of the user within the environment (e.g., for adjusting the light intensity, chromaticity, temperature, sound level, etc. of the environment)).

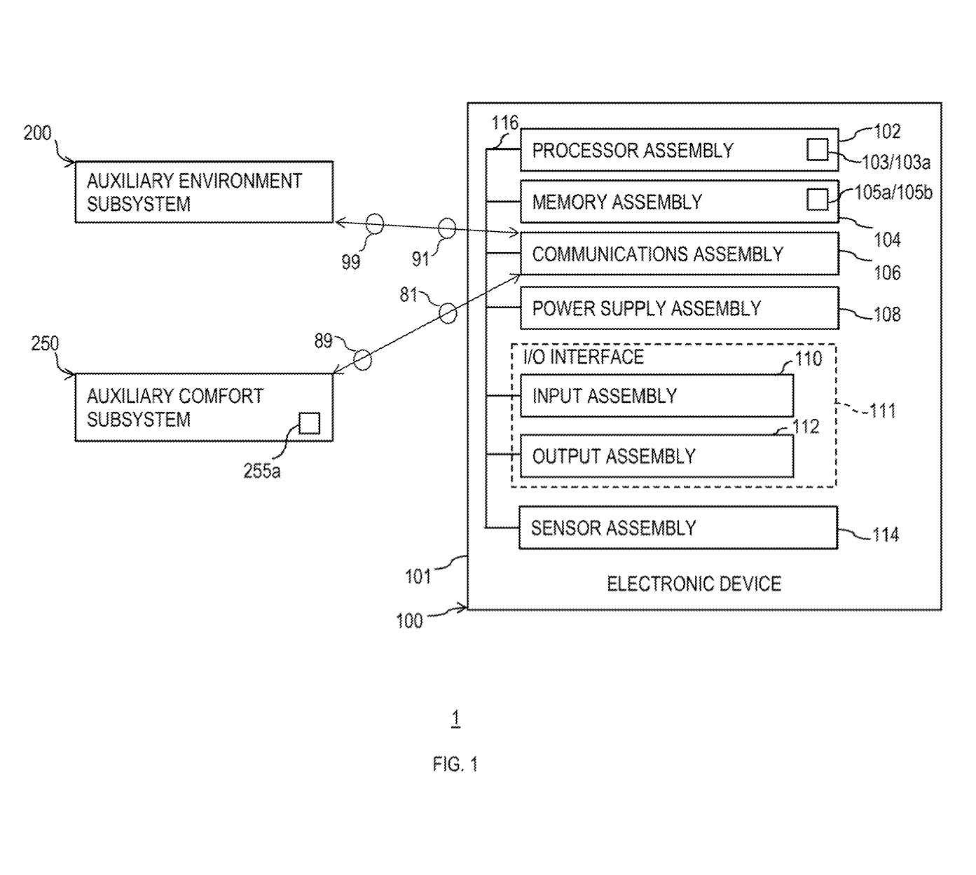

FIG. 1 is a schematic view of an illustrative system 1 that includes an electronic device 100 for managing comfort states in accordance with some embodiments. Electronic device 100 can include, but is not limited to, a music player (e.g., an iPod™ available by Apple Inc. of Cupertino, Calif.), video player, still image player, game player, other media player, music recorder, movie or video camera or recorder, still camera, other media recorder, radio, medical equipment, domestic appliance, transportation vehicle instrument, musical instrument, calculator, cellular telephone (e.g., an iPhone™ available by Apple Inc.), other wireless communication device, wearable device (e.g., an Apple Watch™ available by Apple Inc.), personal digital assistant, remote control, pager, computer (e.g., a desktop (e.g., an iMac™ available by Apple Inc.), laptop (e.g., a MacBook™ available by Apple Inc.), tablet (e.g., an iPad™ available by Apple Inc.), server, etc.), monitor, television, stereo equipment, set up box, set-top box, boom box, modem, router, printer, appliance, security device, or any combination thereof. In some embodiments, electronic device 100 may perform a single function (e.g., a device dedicated to determining a comfort level of a user) and, in other embodiments, electronic device 100 may perform multiple functions (e.g., a device that determines a comfort level of a user, plays music, and receives and transmits telephone calls). Electronic device 100 may be any portable, mobile, hand-held, or miniature electronic device that may be configured to determine a comfort level of a user wherever the user travels. Some miniature electronic devices may have a form factor that is smaller than that of hand-held electronic devices, such as an iPod™. Illustrative miniature electronic devices can be integrated into various objects that may include, but are not limited to, watches (e.g., an Apple Watch™ available by Apple Inc.), rings, necklaces, belts, accessories for belts, headsets, accessories for shoes, virtual reality devices, glasses, other wearable electronics, accessories for sporting equipment, accessories for fitness equipment, key chains, or any combination thereof. Alternatively, electronic device 100 may not be portable at all, but may instead be generally stationary.

As shown in FIG. 1, for example, electronic device 100 may include a processor assembly 102, a memory assembly 104, a communications assembly 106, a power supply assembly 108, an input assembly 110, an output assembly 112, and a sensor assembly 114. Electronic device 100 may also include a bus 116 that may provide one or more wired or wireless communication links or paths for transferring data and/or power to, from, or between various assemblies of electronic device 100. In some embodiments, one or more assemblies of electronic device 100 may be combined or omitted. Moreover, electronic device 100 may include any other suitable assemblies not combined or included in FIG. 1 and/or several instances of the assemblies shown in FIG. 1. For the sake of simplicity, only one of each of the assemblies is shown in FIG. 1.

Memory assembly 104 may include one or more storage mediums, including for example, a hard-drive, flash memory, permanent memory such as read-only memory (“ROM”), semi-permanent memory such as random access memory (“RAM”), any other suitable type of storage assembly, or any combination thereof. Memory assembly 104 may include cache memory, which may be one or more different types of memory used for temporarily storing data for electronic device applications. Memory assembly 104 may be fixedly embedded within electronic device 100 or may be incorporated onto one or more suitable types of components that may be repeatedly inserted into and removed from electronic device 100 (e.g., a subscriber identity module (“SIM”) card or secure digital (“SD”) memory card). Memory assembly 104 may store media data (e.g., music and image files), software (e.g., for implementing functions on device 100), firmware, preference information (e.g., media playback preferences), lifestyle information (e.g., food preferences), exercise information (e.g., information obtained by exercise monitoring applications), sleep information (e.g., information obtained by sleep monitoring applications), mindfulness information (e.g., information obtained by mindfulness monitoring applications), transaction information (e.g., credit card information), wireless connection information (e.g., information that may enable device 100 to establish a wireless connection), subscription information (e.g., information that keeps track of podcasts or television shows or other media a user subscribes to), contact information (e.g., telephone numbers and e-mail addresses), calendar information, pass information (e.g., transportation boarding passes, event tickets, coupons, store cards, financial payment cards, etc.), any suitable device comfort model data of device 100 (e.g., as may be stored in any suitable device comfort model 105a of memory assembly 104), any suitable environmental behavior data 105b of memory assembly 104, any other suitable data, or any combination thereof.

Communications assembly 106 may be provided to allow device 100 to communicate with one or more other electronic devices or servers or subsystems or any other entities remote from device 100 (e.g., one or more of auxiliary subsystems 200 and 250 of system 1 of FIG. 1) using any suitable communications protocol(s). For example, communications assembly 106 may support Wi-Fi™ (e.g., an 802.11 protocol), ZigBee™ (e.g., an 802.15.4 protocol), WiDi™, Ethernet, Bluetooth™, Bluetooth™ Low Energy (“BLE”), high frequency systems (e.g., 900 MHz, 2.4 GHz, and 5.6 GHz communication systems), infrared, transmission control protocol/internet protocol (“TCP/IP”) (e.g., any of the protocols used in each of the TCP/IP layers), Stream Control Transmission Protocol (“SCTP”), Dynamic Host Configuration Protocol (“DHCP”), hypertext transfer protocol (“HTTP”), BitTorrent™, file transfer protocol (“FTP”), real-time transport protocol (“RTP”), real-time streaming protocol (“RTSP”), real-time control protocol (“RTCP”), Remote Audio Output Protocol (“RAOP”), Real Data Transport Protocol™ (“RDTP”), User Datagram Protocol (“UDP”), secure shell protocol (“SSH”), wireless distribution system (“WDS”) bridging, any communications protocol that may be used by wireless and cellular telephones and personal e-mail devices (e.g., Global System for Mobile Communications (“GSM”), GSM plus Enhanced Data rates for GSM Evolution (“EDGE”), Code Division Multiple Access (“CDMA”), Orthogonal Frequency-Division Multiple Access (“OFDMA”), high speed packet access (“HSPA”), multi-band, etc.), any communications protocol that may be used by a low power Wireless Personal Area Network (“6LoWPAN”) module, any other communications protocol, or any combination thereof. Communications assembly 106 may also include or may be electrically coupled to any suitable transceiver circuitry that can enable device 100 to be communicatively coupled to another device (e.g., a server, host computer, scanner, accessory device, subsystem, etc.) and communicate data with that other device wirelessly or via a wired connection (e.g., using a connector port). Communications assembly 106 (and/or sensor assembly 114) may be configured to determine a geographical position of electronic device 100 and/or any suitable data that may be associated with that position. For example, communications assembly 106 may utilize a global positioning system (“GPS”) or a regional or site-wide positioning system that may use cell tower positioning technology or Wi-Fi™ technology, or any suitable location-based service or real-time locating system, which may use a geo-fence for providing any suitable location-based data to device 100 (e.g., to determine a current geo-location of device 100 and/or any other suitable associated data (e.g., the current location is a library, the current location is outside, the current location is your home, etc.)).

Power supply assembly 108 may include any suitable circuitry for receiving and/or generating power, and for providing such power to one or more of the other assemblies of electronic device 100. For example, power supply assembly 108 can be coupled to a power grid (e.g., when device 100 is not acting as a portable device or when a battery of the device is being charged at an electrical outlet with power generated by an electrical power plant). As another example, power supply assembly 108 may be configured to generate power from a natural source (e.g., solar power using solar cells). As another example, power supply assembly 108 can include one or more batteries for providing power (e.g., when device 100 is acting as a portable device).

One or more input assemblies 110 may be provided to permit a user or device environment to interact or interface with device 100. For example, input assembly 110 can take a variety of forms, including, but not limited to, a touch pad, dial, click wheel, scroll wheel, touch screen, one or more buttons (e.g., a keyboard), mouse, joy stick, track ball, microphone, camera, scanner (e.g., a barcode scanner or any other suitable scanner that may obtain product identifying information from a code, such as a linear barcode, a matrix barcode (e.g., a quick response (“QR”) code), or the like), proximity sensor, light detector, temperature sensor, motion sensor, biometric sensor (e.g., a fingerprint reader or other feature (e.g., facial) recognition sensor, which may operate in conjunction with a feature-processing application that may be accessible to electronic device 100 for authenticating a user), line-in connector for data and/or power, and combinations thereof. Each input assembly 110 can be configured to provide one or more dedicated control functions for making selections or issuing commands associated with operating device 100. Each input assembly 110 may be positioned at any suitable location at least partially within a space defined by a housing 101 of device 100 and/or at least partially on an external surface of housing 101 of device 100.

Electronic device 100 may also include one or more output assemblies 112 that may present information (e.g., graphical, audible, and/or tactile information) to a user of device 100. For example, output assembly 112 of electronic device 100 may take various forms, including, but not limited to, audio speakers, headphones, line-out connectors for data and/or power, visual displays (e.g., for transmitting data via visible light and/or via invisible light), infrared ports, flashes (e.g., light sources for providing artificial light for illuminating an environment of the device), tactile/haptic outputs (e.g., rumblers, vibrators, etc.), and combinations thereof. As a specific example, electronic device 100 may include a display assembly output assembly as output assembly 112, where such a display assembly output assembly may include any suitable type of display or interface for presenting visual data to a user with visible light.

It is noted that one or more input assemblies and one or more output assemblies may sometimes be referred to collectively herein as an input/output (“I/O”) assembly or I/O interface (e.g., input assembly 110 and output assembly 112 as I/O assembly or user interface assembly or I/O interface 111). For example, input assembly 110 and output assembly 112 may sometimes be a single I/O interface 111, such as a touch screen, that may receive input information through a user's touch of a display screen and that may also provide visual information to a user via that same display screen.

Sensor assembly 114 may include any suitable sensor or any suitable combination of sensors operative to detect movements of electronic device 100 and/or of a user thereof and/or any other characteristics of device 100 and/or of its environment (e.g., physical activity or other characteristics of a user of device 100, light content of the device environment, gas pollution content of the device environment, noise pollution content of the device environment, etc.). Sensor assembly 114 may include any suitable sensor(s), including, but not limited to, one or more of a GPS sensor, accelerometer, directional sensor (e.g., compass), gyroscope, motion sensor, pedometer, passive infrared sensor, ultrasonic sensor, microwave sensor, a tomographic motion detector, a camera, a biometric sensor, a light sensor, a timer, or the like.

Sensor assembly 114 may include any suitable sensor components or subassemblies for detecting any suitable movement of device 100 and/or of a user thereof. For example, sensor assembly 114 may include one or more three-axis acceleration motion sensors (e.g., an accelerometer) that may be operative to detect linear acceleration in three directions (i.e., the x- or left/right direction, the y- or up/down direction, and the z- or forward/backward direction). As another example, sensor assembly 114 may include one or more single-axis or two-axis acceleration motion sensors that may be operative to detect linear acceleration only along each of the x- or left/right direction and the y- or up/down direction, or along any other pair of directions. In some embodiments, sensor assembly 114 may include an electrostatic capacitance (e.g., capacitance-coupling) accelerometer that may be based on silicon micro-machined micro electro-mechanical systems (“MEMS”) technology, including a heat-based MEMS type accelerometer, a piezoelectric type accelerometer, a piezo-resistance type accelerometer, and/or any other suitable accelerometer (e.g., which may provide a pedometer or other suitable function). Sensor assembly 114 may be operative to directly or indirectly detect rotation, rotational movement, angular displacement, tilt, position, orientation, motion along a non-linear (e.g., arcuate) path, or any other non-linear motions. Additionally or alternatively, sensor assembly 114 may include one or more angular rate, inertial, and/or gyro-motion sensors or gyroscopes for detecting rotational movement. For example, sensor assembly 114 may include one or more rotating or vibrating elements, optical gyroscopes, vibrating gyroscopes, gas rate gyroscopes, ring gyroscopes, magnetometers (e.g., scalar or vector magnetometers), compasses, and/or the like. Any other suitable sensors may also or alternatively be provided by sensor assembly 114 for detecting motion on device 100, such as any suitable pressure sensors, altimeters, or the like. Using sensor assembly 114, electronic device 100 may be configured to determine a velocity, acceleration, orientation, and/or any other suitable motion attribute of electronic device 100.

Sensor assembly 114 may include any suitable sensor components or subassemblies for detecting any suitable biometric data and/or health data and/or sleep data and/or mindfulness data and/or the like of a user of device 100. For example, sensor assembly 114 may include any suitable biometric sensor that may include, but is not limited to, one or more health-related optical sensors, capacitive sensors, thermal sensors, electric field (“eField”) sensors, and/or ultrasound sensors, such as photoplethysmogram (“PPG”) sensors, electrocardiography (“ECG”) sensors, galvanic skin response (“GSR”) sensors, posture sensors, stress sensors, photoplethysmogram sensors, and/or the like. These sensors can generate data providing health-related information associated with the user. For example, PPG sensors can provide information regarding a user's respiratory rate, blood pressure, and/or oxygen saturation. ECG sensors can provide information regarding a user's heartbeats. GSR sensors can provide information regarding a user's skin moisture, which may be indicative of sweating and can prioritize a thermostat application to determine a user's body temperature. In some examples, each sensor can be a separate device, while, in other examples, any combination of two or more of the sensors can be included within a single device. For example, a gyroscope, accelerometer, photoplethysmogram, galvanic skin response sensor, and temperature sensor can be included within a wearable electronic device, such as a smart watch, while a scale, blood pressure cuff, blood glucose monitor, SpO2 sensor, respiration sensor, posture sensor, stress sensor, and asthma inhaler can each be separate devices. While specific examples are provided, it should be appreciated that other sensors can be used and other combinations of sensors can be combined into a single device. Using one or more of these sensors, device 100 can determine physiological characteristics of the user while performing a detected activity, such as a heart rate of a user associated with the detected activity, average body temperature of a user detected during the detected activity, any normal or abnormal physical conditions associated with the detected activity, or the like. In some examples, a GPS sensor or any other suitable location detection component(s) of device 100 can be used to determine a user's location (e.g., geo-location and/or address and/or location type (e.g., library, school, office, zoo, etc.) and movement, as well as a displacement of the user's motion. An accelerometer, directional sensor, and/or gyroscope can further generate activity data that can be used to determine whether a user of device 100 is engaging in an activity, is inactive, or is performing a gesture. Any suitable activity of a user may be tracked by sensor assembly 114, including, but not limited to, steps taken, flights of stairs climbed, calories burned, distance walked, distance run, minutes of exercise performed and exercise quality, time of sleep and sleep quality, nutritional intake (e.g., foods ingested and their nutritional value), mindfulness activities and quantity and quality thereof (e.g., reading efficiency, data retention efficiency), any suitable work accomplishments of any suitable type (e.g., as may be sensed or logged by user input information indicative of such accomplishments), and/or the like. Device 100 can further include a timer that can be used, for example, to add time dimensions to various attributes of the detected physical activity, such as a duration of a user's physical activity or inactivity, time(s) of a day when the activity is detected or not detected, and/or the like.

Sensor assembly 114 may include any suitable sensor components or subassemblies for detecting any suitable characteristics of any suitable condition of the lighting of the environment of device 100. For example, sensor assembly 114 may include any suitable light sensor that may include, but is not limited to, one or more ambient visible light color sensors, illuminance ambient light level sensors, ultraviolet (“UV”) index and/or UV radiation ambient light sensors, and/or the like. Any suitable light sensor or combination of light sensors may be provided for determining the illuminance or light level of ambient light in the environment of device 100 (e.g., in lux or lumens per square meter, etc.) and/or for determining the ambient color or white point chromaticity of ambient light in the environment of device 100 (e.g., in hue and colorfulness or in x/y parameters with respect to an x-y chromaticity space, etc.) and/or for determining the UV index or UV radiation in the environment of device 100 (e.g., in UV index units, etc.). A suitable light sensor may include, for example, a photodiode, a phototransistor, an integrated photodiode and amplifier, or any other suitable photo-sensitive device. In some embodiments, more than one light sensor may be integrated into device 100. For example, multiple narrowband light sensors may be integrated into device 100 and each light sensor may be sensitive in a different portion of the light spectrum (e.g., three narrowband light sensors may be integrated into a single sensor package: a first light sensor may be sensitive to light in the red region of the electromagnetic spectrum; a second light sensor may be sensitive in a blue region of the electromagnetic spectrum; and a third light sensor may be sensitive in the green portion of the electromagnetic spectrum). Additionally or alternatively, one or more broadband light sensors may be integrated into device 100. The sensing frequencies of each narrowband sensor may also partially overlap, or nearly overlap, that of another narrowband sensor. Each of the broadband light sensors may be sensitive to light throughout the spectrum of visible light and the various ranges of visible light (e.g., red, green, and blue ranges) may be filtered out so that a determination may be made as to the color of the ambient light. As used herein, “white point” may refer to coordinates in a chromaticity curve that may define the color “white.” For example, a plot of a chromaticity curve from the Commission International de l'Eclairage (“CIE”) may be accessible to system 1 (e.g., as a portion of data stored by memory assembly 104), wherein the circumference of the chromaticity curve may represent a range of wavelengths in nanometers of visible light and, hence, may represent true colors, whereas points contained within the area defined by the chromaticity curve may represent a mixture of colors. A Planckian curve may be defined within the area defined by the chromaticity curve and may correspond to colors of a black body when heated. The Planckian curve passes through a white region (i.e., the region that includes a combination of all the colors) and, as such, the term “white point” is sometimes generalized as a point along the Planckian curve resulting in either a bluish white point or a yellowish white point. However, “white point” may also include points that are not on the Planckian curve. For example, in some cases the white point may have a reddish hue, a greenish hue, or a hue resulting from any combination of colors. The perceived white point of light sources may vary depending on the ambient lighting conditions in which the lights source is operating. Such a chromaticity curve plot may be used in coordination with any sensed light characteristics to determine the ambient color (e.g., true color) and/or white point chromaticity of the environment of device 100 in any suitable manner. Any suitable UV index sensors and/or ambient color sensors and/or illuminance sensors may be provided by sensor assembly 114 in order to determine the current UV index and/or chromaticity and/or illuminance of the ambient environment of device 100.

Sensor assembly 114 may include any suitable sensor components or subassemblies for detecting any suitable characteristics of any suitable condition of the air quality of the environment of device 100. For example, sensor assembly 114 may include any suitable air quality sensor that may include, but is not limited to, one or more ambient air flow or air velocity meters, ambient oxygen level sensors, volatile organic compound (“VOC”) sensors, ambient humidity sensors, ambient temperature sensors, and/or the like. Any suitable ambient air sensor or combination of ambient air sensors may be provided for determining the oxygen level of the ambient air in the environment of device 100 (e.g., in O₂% per liter, etc.) and/or for determining the air velocity of the ambient air in the environment of device 100 (e.g., in kilograms per second, etc.) and/or for determining the level of any suitable harmful gas or potentially harmful substance (e.g., VOC (e.g., any suitable harmful gasses, scents, odors, etc.) or particulate or dust or pollen or mold or the like) of the ambient air in the environment of device 100 (e.g., in HG % per liter, etc.) and/or for determining the humidity of the ambient air in the environment of device 100 (e.g., in grams of water per cubic meter, etc. (e.g., using a hygrometer)) and/or for determining the temperature of the ambient air in the environment of device 100 (e.g., in degrees Celsius, etc. (e.g., using a thermometer)).

Sensor assembly 114 may include any suitable sensor components or subassemblies for detecting any suitable characteristics of any suitable condition of the sound quality of the environment of device 100. For example, sensor assembly 114 may include any suitable sound quality sensor that may include, but is not limited to, one or more microphones or the like that may determine the level of sound pollution or noise in the environment of device 100 (e.g., in decibels, etc.). Sensor assembly 114 may also include any other suitable sensor for determining any other suitable characteristics about a user of device 100 and/or the environment of device 100 and/or any situation within which device 100 may be existing. For example, any suitable clock and/or position sensor(s) may be provided to determine the current time and/or time zone within which device 100 may be located.

One or more sensors of sensor assembly 114 may be embedded in a body (e.g., housing 101) of device 100, such as along a bottom surface that may be operative to contact a user, or can be positioned at any other desirable location. In some examples, different sensors can be placed in different locations inside or on the surfaces of device 100 (e.g., some located inside housing 101 and some attached to an attachment mechanism (e.g., a wrist band coupled to a housing of a wearable device), or the like). In other examples, one or more sensors can be worn by a user separately as different parts of a single device 100 or as different devices. In such cases, the sensors can be configured to communicate with device 100 using a wired and/or wireless technology (e.g., via communications assembly 106). In some examples, sensors can be configured to communicate with each other and/or share data collected from one or more sensors. In some examples, device 100 can be waterproof such that the sensors can detect a user's activity in water.

System 1 may include one or more auxiliary environment subsystems 200 that may include any suitable assemblies, such as assemblies that may be similar to one, some, or each of the assemblies of device 100. Subsystem 200 may be configured to communicate any suitable auxiliary environment subsystem data 91 to device 100 (e.g., via a communications assembly of subsystem 200 and communications assembly 106 of device 100), such as automatically and/or in response to an auxiliary environment subsystem data request of data 99 that may be communicated from device 100 to auxiliary environment subsystem 200. Such auxiliary environment subsystem data 91 may be any suitable environmental attribute data that may be indicative of any suitable condition(s) of the environment of subsystem 200 as may be detected by auxiliary environment subsystem 200 (e.g., as may be detected by any suitable input assembly and/or any suitable sensor assembly of auxiliary environment subsystem 200) and/or any suitable subsystem state data that may be indicative of the current state of any components/features of auxiliary environment subsystem 200 (e.g., any state of any suitable output assembly and/or of any suitable application of auxiliary environment subsystem 200) and/or any suitable subsystem functionality data that may be indicative of any suitable functionalities/capabilities of auxiliary environment subsystem 200. In some embodiments, such communicated auxiliary environment subsystem data 91 may be indicative of any suitable characteristic of an environment of auxiliary environment subsystem 200 that may be an environment shared by device 100. For example, subsystem 200 may include any suitable sensor assembly with any suitable sensors that may be operative to determine any suitable characteristic of an environment of subsystem 200, which may be positioned in an environment shared by device 100. As just one example, subsystem 200 may include or may be in communication with a heating, ventilation, and air conditioning (“HVAC”) subsystem of an environment, and device 100 may be able to access any suitable HVAC data (e.g., any suitable auxiliary environment subsystem data 91) from auxiliary environment subsystem 200 indicative of any suitable HVAC characteristics (e.g., temperature, humidity, air velocity, oxygen level, harmful gas level, etc.) of the environment, such as when device 100 is located within that environment. As just one other example, subsystem 200 may include or may be in communication with a lighting subsystem of an environment, and device 100 may be able to access any suitable lighting data (e.g., any suitable auxiliary environment subsystem data 91) from auxiliary environment subsystem 200 indicative of any suitable lighting characteristics (e.g., brightness, color, etc.) emitted by subsystem 200 and/or capable of being emitted by subsystem 200. As yet just one other example, subsystem 200 may include or may be in communication with a sound subsystem of an environment, and device 100 may be able to access any suitable sound data (e.g., any suitable auxiliary environment subsystem data 91) from auxiliary environment subsystem 200 indicative of any suitable sound characteristics (e.g., volume, frequency characteristics, etc.) emitted by subsystem 200 and/or capable of being emitted by subsystem 200. As yet just one other example, subsystem 200 may be provided by a weather service (e.g., a subsystem operated by a local weather service or a national or international weather service) that may be operative to determine the weather (e.g., temperature, humidity, gas levels, air velocity, etc.) for any suitable environment (e.g., at least any outdoor environment). It is to be understood that auxiliary environment subsystem 200 may be any suitable subsystem that may be operative to determine or generate and/or control and/or access any suitable environmental data about a particular environment and share such data (e.g., as any suitable auxiliary environment subsystem data 91) with device 100 at any suitable time, such as to augment and/or enhance the environmental sensing capabilities of sensor assembly 114 of device 100. Electronic device 100 may be operative to communicate any suitable data 99 from communications assembly 106 to a communications assembly of auxiliary environment subsystem 200 using any suitable communication protocol(s), where such data 99 may be any suitable request data for instructing subsystem 200 to share data 91 and/or may be any suitable auxiliary environment subsystem control data that may be operative to adjust any physical system attributes of auxiliary environment subsystem 200 (e.g., of any suitable output assembly of auxiliary environment subsystem 200 (e.g., to increase the temperature of air output by an HVAC auxiliary environment subsystem 200, to adjust the light being emitted by a light auxiliary environment subsystem 200, to adjust the sound being emitted by a sound auxiliary environment subsystem 200, etc.)).

Device 100 may be situated in various environments at various times (e.g., outdoors in a park at 11:00 AM, indoors in a library at 2:00 PM, outdoors on a city sidewalk at 5:00 PM, indoors in a restaurant at 9:00 PM, etc.). At any particular environment in which device 100 may be situated at a particular time, any or all environmental characteristic information indicative of the particular environment at the particular time may be sensed by device 100 from any or all features (e.g., people, animals, machines, light sources, sound sources, etc.) of the environment (e.g., directly via sensor assembly 114 of device 100 and/or via any suitable auxiliary environment subsystem(s) 200 of the environment). Such environmental characteristic information that may be sensed or otherwise received by device 100 for a particular environment at a particular time may be processed and/or stored by device 100 as at least a portion of environmental behavior data 105b alone or in conjunction with any suitable user behavior information that may be provided by user U (e.g., by input assembly 110) or otherwise detected by device 100 (e.g., by sensor assembly 114) and that may be indicative of a user's behavior within and/or a user's reaction to the particular environment, for example, as at least another portion of environmental behavior data 105b. Any suitable user behavior information for a user at a particular environment at a particular time may be detected in any suitable manner by device 100 (e.g., any suitable user-provided feedback information may be provided by user U to device 100 (e.g., via any suitable input assembly 110 (e.g., typed via a keyboard or dictated via a user microphone, etc.) or detected via any suitable sensor assembly or otherwise of device 100 or a subsystem 200 of the environment) that may be indicative of the user's comfort level in the particular environment at the particular time (e.g., a subjective user-provided ranking, a subjective user-provided preference for adjusting the environment in some way, and/or the like) and/or that may be indicative of the user's performance of any suitable activity in the particular environment at the particular time (e.g., any suitable exercise activity information, any suitable sleep information, any suitable mindfulness information, etc. (e.g., which may be indicative of the user's effectiveness or ability to perform an activity within the particular environment))). Such environmental characteristic information that may be sensed or otherwise received by device 100 for a particular environment at a particular time, as well as such user behavior information that may be sensed or otherwise received by device 100 for the particular environment at the particular time, may together be processed and/or stored by device 100 as at least a portion of environmental behavior data 105b (e.g., for tracking a user's subjective comfort level for a particular environment at a particular time and/or a user's objective activity performance capability for a particular environment at a particular time). Additionally or alternatively, environmental behavior data 105b may include any suitable user environmental preferences that may be provided by a user or otherwise deduced, such as a preferred temperature and/or a preferred noise level and/or the like (e.g., generally or for a particular type of user activity), where such user environmental preference(s) of environmental behavior data 105b may not be associated with a particular environment at a particular time (e.g., unlike user behavior information of environmental behavior data 105b).

Processor assembly 102 of electronic device 100 may include any processing circuitry that may be operative to control the operations and performance of one or more assemblies of electronic device 100. For example, processor assembly 102 may receive input signals from input assembly 110 and/or drive output signals through output assembly 112. As shown in FIG. 1, processor assembly 102 may be used to run one or more applications, such as an application 103. Application 103 may include, but is not limited to, one or more operating system applications, firmware applications, media playback applications, media editing applications, pass applications, calendar applications, state determination applications, biometric feature-processing applications, compass applications, health applications, mindfulness applications, sleep applications, thermometer applications, weather applications, thermal management applications, video game applications, comfort applications, device and/or user activity applications, or any other suitable applications. For example, processor assembly 102 may load application 103 as a user interface program to determine how instructions or data received via an input assembly 110 and/or sensor assembly 114 and/or any other assembly of device 100 (e.g., any suitable auxiliary environment subsystem data 99 that may be received by device 100 via communications assembly 106) may manipulate the one or more ways in which information may be stored on device 100 and/or provided to a user via an output assembly 112 and/or provided to an auxiliary environment subsystem (e.g., to subsystem 200 as auxiliary environment subsystem data 91 via communications assembly 106). Application 103 may be accessed by processor assembly 102 from any suitable source, such as from memory assembly 104 (e.g., via bus 116) or from another remote device or server (e.g., from a subsystem 200 and/or from a subsystem 250 of system 1 via communications assembly 106). Processor assembly 102 may include a single processor or multiple processors. For example, processor assembly 102 may include at least one “general purpose” microprocessor, a combination of general and special purpose microprocessors, instruction set processors, graphics processors, video processors, and/or related chips sets, and/or special purpose microprocessors. Processor assembly 102 also may include on board memory for caching purposes.

One particular type of application available to processor assembly 102 may be an activity application 103a that may be operative to determine or predict a current or planned activity of device 100 and/or for a user thereof. Such an activity may be determined by activity application 103a based on any suitable data accessible by activity application 103a (e.g., from memory assembly 104 and/or from any suitable remote entity (e.g., any suitable auxiliary environment subsystem data 91 from any suitable auxiliary subsystem 200 via communications assembly 106)), such as data from any suitable activity data source, including, but not limited to, a calendar application, a health application, a social media application, an exercise monitoring application, a sleep monitoring application, a mindfulness monitoring application, transaction information, wireless connection information, subscription information, contact information, pass information, current environmental behavior data 105b, previous environmental behavior data 105b, comfort model data of any suitable comfort model, and/or the like. For example, at a particular time, such an activity application 103a may be operative to determine one or more potential or planned or predicted activities for that particular time, such as exercise, sleep, eat, study, read, relax, play, and/or the like. Alternatively, such an activity application 103a may request that a user indicate a planned activity (e.g., via a user interface assembly).

Electronic device 100 may also be provided with housing 101 that may at least partially enclose at least a portion of one or more of the assemblies of device 100 for protection from debris and other degrading forces external to device 100. In some embodiments, one or more of the assemblies may be provided within its own housing (e.g., input assembly 110 may be an independent keyboard or mouse within its own housing that may wirelessly or through a wire communicate with processor assembly 102, which may be provided within its own housing).

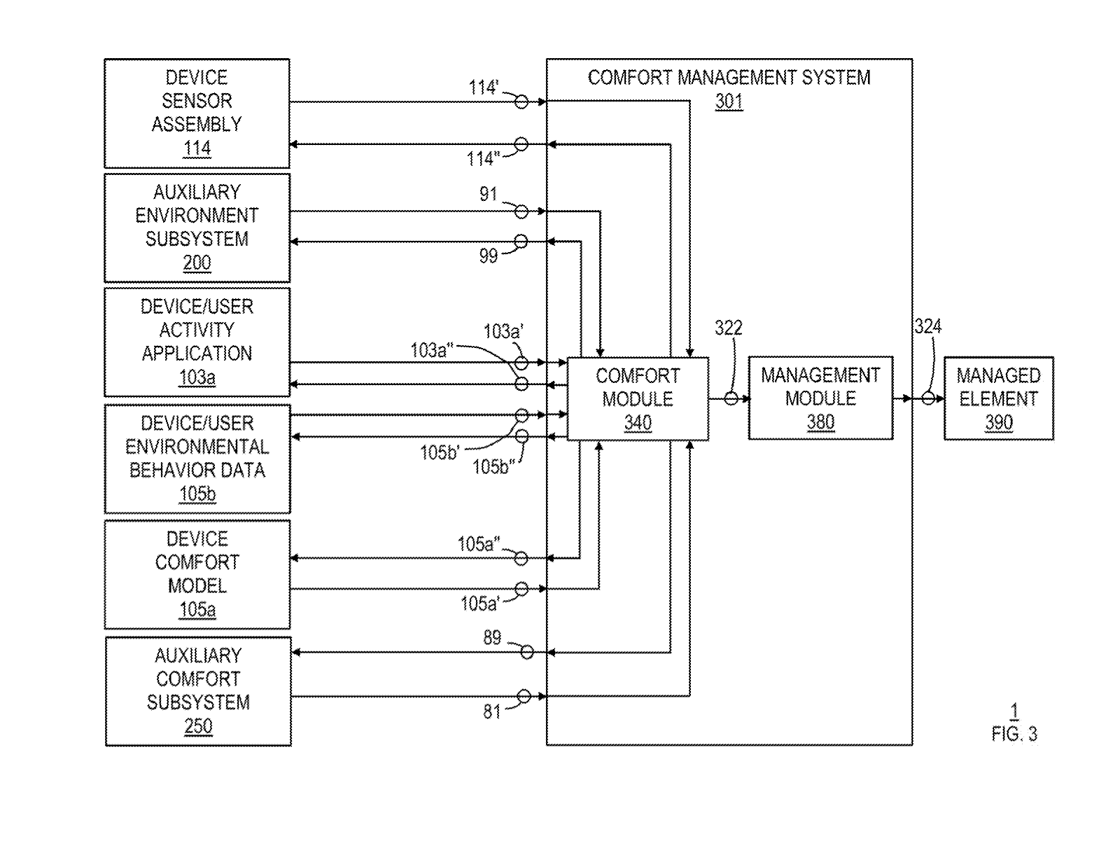

Processor assembly 102 may load any suitable application 103 as a background application program or a user-detectable application program in conjunction with any suitable comfort model to determine how any suitable input assembly data received via any suitable input assembly 110 and/or any suitable sensor assembly data received via any suitable sensor assembly 114 and/or any other suitable data received via any other suitable assembly of device 100 (e.g., any suitable auxiliary environment subsystem data 91 received from auxiliary environment subsystem 200 via communications assembly 106 of device 100 and/or any suitable planned activity data as may be determined by activity application 103a of device 100) may be used to determine any suitable comfort state data (e.g., comfort state data 322 of FIG. 3) that may be used to control or manipulate at least one functionality of device 100 (e.g., a performance or mode of electronic device 100 that may be altered in a particular one of various ways (e.g., particular comfort alerts or recommendations may be provided to a user via a user interface assembly and/or particular comfort adjustments may be made by an output assembly and/or the like)). Any suitable comfort model or any suitable combination of two or more comfort models may be used by device 100 in order to make any suitable comfort state determination for any particular environment of device 100 at any particular time (e.g., any comfort model(s) may be used in conjunction with any suitable environmental behavior data 105b (e.g., any suitable environmental characteristic information and/or any suitable user behavior information that may be sensed or otherwise received by device 100) and/or in conjunction with any suitable planned activity (e.g., any suitable activity as may be determined by activity application 103a) to provide any suitable comfort state data that may be indicative of any comfort level determination for the particular environment at the particular time). For example, a device comfort model 105a may be maintained and updated on device 100 (e.g., in memory assembly 104) using processing capabilities of processor assembly 102. Additionally or alternatively, an auxiliary comfort model 255a may be maintained and updated by any suitable auxiliary comfort subsystem 250 that may include any suitable assemblies, such as assemblies that may be similar to one, some, or each of the assemblies of device 100. Auxiliary comfort subsystem 250 may be configured to communicate any suitable auxiliary comfort subsystem data 81 to device 100 (e.g., via a communications assembly of subsystem 250 and communications assembly 106 of device 100), such as automatically and/or in response to an auxiliary comfort subsystem data request of data 89 that may be communicated from device 100 to auxiliary comfort subsystem 250. Such auxiliary comfort subsystem data 81 may be any suitable portion or the entirety of auxiliary comfort model 255a for use by device 100 (e.g., for use by an application 103 instead of or in addition to (e.g., as a supplement to) device comfort model 105a).

A comfort model may be developed and/or generated for use in evaluating and/or predicting a comfort state for a particular environment (e.g., at a particular time and/or with respect to one or more particular activities). For example, a comfort model may be a learning engine for an experiencing entity (e.g., a particular user or a particular subset or type of user or all users generally), where the learning engine may be operative to use any suitable machine learning to use certain environment data (e.g., one or more various types or categories of environment category data, such as environmental behavior data (e.g., environmental characteristic information and/or user behavior information) and/or planned activity data) for a particular environment (e.g., at a particular time and/or with respect to one or more planned activities) in order to predict, estimate, and/or otherwise generate a comfort score and/or any suitable comfort state determination that may be indicative of the comfort that may be experienced at the particular environment by the experiencing entity (e.g., a comfort level that may be derived by the user at the environment). For example, the learning engine may include any suitable neural network (e.g., an artificial neural network) that may be initially configured, trained on one or more sets of scored environment data from any suitable experiencing entity(ies), and then used to predict a comfort score or any other suitable comfort state determination based on another set of environment data.

A neural network or neuronal network or artificial neural network may be hardware-based, software-based, or any combination thereof, such as any suitable model (e.g., an analytical model, a computational model, etc.), which, in some embodiments, may include one or more sets or matrices of weights (e.g., adaptive weights, which may be numerical parameters that may be tuned by one or more learning algorithms or training methods or other suitable processes) and/or may be capable of approximating one or more functions (e.g., non-linear functions or transfer functions) of its inputs. The weights may be connection strengths between neurons of the network, which may be activated during training and/or prediction. A neural network may generally be a system of interconnected neurons that can compute values from inputs and/or that may be capable of machine learning and/or pattern recognition (e.g., due to an adaptive nature). A neural network may use any suitable machine learning techniques to optimize a training process. The neural network may be used to estimate or approximate functions that can depend on a large number of inputs and that may be generally unknown. The neural network may generally be a system of interconnected “neurons” that may exchange messages between each other, where the connections may have numeric weights (e.g., initially configured with initial weight values) that can be tuned based on experience, making the neural network adaptive to inputs and capable of learning (e.g., learning pattern recognition). A suitable optimization or training process may be operative to modify a set of initially configured weights assigned to the output of one, some, or all neurons from the input(s) and/or hidden layer(s). A non-linear transfer function may be used to couple any two portions of any two layers of neurons, including an input layer, one or more hidden layers, and an output (e.g., an input to a hidden layer, a hidden layer to an output, etc.).

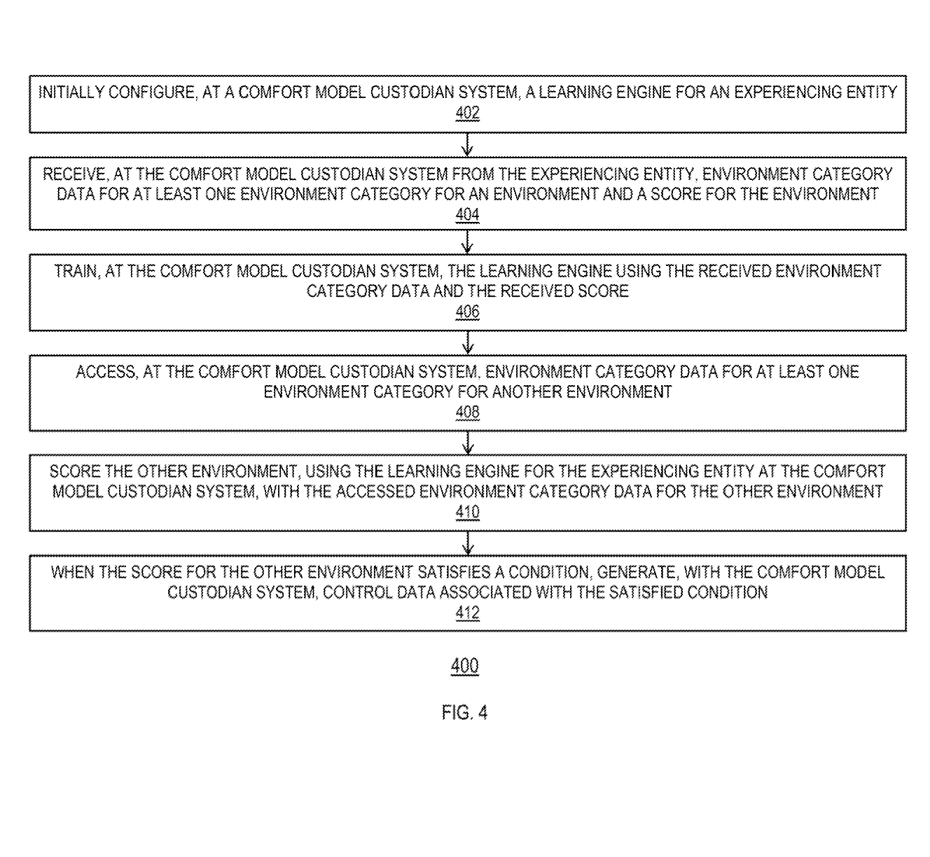

Different input neurons of the neural network may be associated with respective different types of environment categories and may be activated by environment category data of the respective environment categories (e.g., each possible category of environmental characteristic information (e.g., temperature, illuminance/light level, ambient color/white point chromaticity, UV index, noise level, oxygen level, air velocity, humidity, various gas levels (e.g., various VOC levels, pollen level, dust level, etc.), geo-location, location type, time of day, day of week, week of month, week of year, month of year, season, holiday, time zone, and/or the like), each possible category of user behavior information, each possible category of user environmental preferences, and/or each possible category of planned activity (e.g., exercise, read, sleep, study, work, etc.) may be associated with one or more particular respective input neurons of the neural network and environment category data for the particular environment category may be operative to activate the associated input neuron(s)). The weight assigned to the output of each neuron may be initially configured (e.g., at operation 402 of process 400 of FIG. 4) using any suitable determinations that may be made by a custodian or processor of the comfort model (e.g., device 100 and/or auxiliary comfort subsystem 250) based on the data available to that custodian.

The initial configuring of the learning engine or comfort model for the experiencing entity (e.g., the initial weighting and arranging of neurons of a neural network of the learning engine) may be done using any suitable data accessible to a custodian of the comfort model (e.g., a manufacturer of device 100 or of a portion thereof (e.g., device comfort model 105a), any suitable maintenance entity that manages auxiliary comfort subsystem 250, and/or the like), such as data associated with the configuration of other learning engines of system 1 (e.g., learning engines or comfort models for similar experiencing entities), data associated with the experiencing entity (e.g., initial background data accessible by the model custodian about the experiencing entity's composition, background, interests, goals, past experiences, and/or the like), data assumed or inferred by the model custodian using any suitable guidance, and/or the like. For example, a model custodian may be operative to capture any suitable initial background data about the experiencing entity in any suitable manner, which may be enabled by any suitable user interface provided to an appropriate subsystem or device accessible to one, some, or each experiencing entity (e.g., a model app or website). The model custodian may provide a data collection portal for enabling any suitable entity to provide initial background data for the experiencing entity. The data may be uploaded in bulk or manually entered in any suitable manner. In a particular embodiment where the experiencing entity is a particular user or a group of users, the following is a list of just some of the one or more potential types of data that may be collected by a model custodian (e.g., for use in initially configuring the model): sample questions for which answers may be collected may include, but are not limited to, questions related to an experiencing entity's evaluation of perceived comfort with respect to a particular previously experienced environment, their preferred comfort zone (e.g., preferred temperature and/or noise level (e.g., generally and/or for a particular planned activity and/or for a particular type of environment), ideal environment, and/or the like.

A comfort model custodian may receive from the experiencing entity (e.g., at operation 404 of process 400 of FIG. 4) not only environment category data for at least one environment category for an environment that the experiencing entity is currently experiencing or has previously experienced but also a score for that environment experience (e.g., a score that the experiencing entity may supply as an indication of the comfort level that the experiencing entity experienced from experiencing the environment). This may be enabled by any suitable user interface provided to any suitable experiencing entity by any suitable comfort model custodian (e.g., a user interface app or website that may be accessed by the experiencing entity). The comfort model custodian may provide a data collection portal for enabling any suitable entity to provide such data. The score (e.g., comfort score) for the environment may be received and may be derived from the experiencing entity in any suitable manner. For example, a single questionnaire or survey may be provided by the model custodian for deriving not only experiencing entity responses with respect to environment category data for an environment, but also an experiencing entity score for the environment. The model custodian may be configured to provide best practices and standardize much of the evaluation, which may be determined based on the experiencing entity's goals and/or objectives as captured before the environment may have been experience.

A learning engine or comfort model for an experiencing entity may be trained (e.g., at operation 406 of process 400 of FIG. 4) using the received environment category data for the environment (e.g., as inputs of a neural network of the learning engine) and using the received score for the environment (e.g., as an output of the neural network of the learning engine). Any suitable training methods or algorithms (e.g., learning algorithms) may be used to train the neural network of the learning engine, including, but not limited to, Back Propagation, Resilient Propagation, Genetic Algorithms, Simulated Annealing, Levenberg, Nelder-Meade, and/or the like. Such training methods may be used individually and/or in different combinations to get the best performance from a neural network. A loop (e.g., a receipt and train loop) of receiving environment category data and a score for an environment and then training the comfort model using the received environment category data and score (e.g., a loop of operation 404 and operation 406 of process 400 of FIG. 4) may be repeated any suitable number of times for the same experiencing entity and the same learning engine for more effectively training the learning engine for the experiencing entity, where the received environment category data and the received score received of different receipt and train loops may be for different environments or for the same environment (e.g., at different times and/or with respect to different planned activities) and/or may be received from the same source or from different sources of the experiencing entity (e.g., from different users of the experiencing entity) (e.g., a first receipt and train loop may include receiving environment category data and a score from a first user with respect to that user's experience with a first environment, while a second receipt and train loop may include receiving environment category data and a score from a second user with respect to that user's experience with the first environment, while a third receipt and train loop may include receiving environment category data and a score from a third user with respect to that user's experience with a second environment for a planned exercise activity, while a fourth receipt and train loop may include receiving environment category data and a score from a fourth user with respect to that user's experience with the second environment for a planned studying activity, and/or the like), while the training of different receipt and train loops may be done for the same learning engine using whatever environment category data and score was received for the particular receipt and train loop. The number and/or type(s) of the one or more environment categories for which environment category data may be received for one receipt and train loop may be the same or different in any way(s) than the number and/or type(s) of the one or more environment categories for which environment category data may be received for a second receipt and train loop.

A comfort model custodian may access (e.g., at operation 408 of process 400 of FIG. 4) environment category data for at least one environment category for another environment (e.g., an environment that is different than any environment considered at any environment category data receipt of a receipt and train loop for training the learning engine for the experiencing entity). In some embodiments, this other environment may be an environment that has not been specifically experienced by any experiencing entity prior to use of the comfort model in an end user use case. Although, it is to be understood that this other environment may be any suitable environment. The environment category data for this other environment may be accessed from or otherwise provided by any suitable source(s) using any suitable methods (e.g., from one or more sensor assemblies and/or input assemblies of any suitable device(s) 100 and/or subsystem(s) 200 that may be associated with the particular environment at the particular time) for use by the comfort model custodian (e.g., processor assembly 102 of device 100 and/or auxiliary comfort subsystem 250).

This other environment (e.g., environment of interest) may then be scored (e.g., at operation 408 of process 400 of FIG. 4) using the learning engine or comfort model for the experiencing entity with the environment category data accessed for such another environment. For example, the environment category data accessed for the environment of interest may be utilized as input(s) to the neural network of the learning engine (e.g., at operation 410 of process 400 of FIG. 4) similarly to how the environment category data accessed at a receipt portion of a receipt and train loop may be utilized as input(s) to the neural network of the learning engine at a training portion of the receipt and train loop, and such utilization of the learning engine with respect to the environment category data accessed for the environment of interest may result in the neural network providing an output indicative of a comfort score or comfort level or comfort state that may represent the learning engine's predicted or estimated comfort to be derived from the environment of interest by the experiencing entity.

After a comfort score (e.g., any suitable comfort state data (e.g., comfort state data 322 of FIG. 3)) is realized for an environment of interest (e.g., for a current environment being experienced by an experiencing entity (e.g., for a particular time and/or for a particular planned activity)), it may be determined (e.g., at operation 412 of process 400 of FIG. 4) whether the realized score satisfies a particular condition of any suitable number of potential conditions and, if so, the model custodian or any other suitable processor assembly or otherwise (e.g., of device 100 and/or of auxiliary comfort subsystem 250) may generate any suitable control data (e.g., comfort mode data (e.g., comfort mode data 324 of system 301 of FIG. 3)) that may be associated with that satisfied condition for controlling any suitable functionality of any suitable output assembly of device 100 or otherwise (e.g., for adjusting a user interface presentation to a user (e.g., to provide a comfort suggestion or a comfort score)), and/or for controlling any suitable functionality of any suitable output assembly of auxiliary environment subsystem 200 or otherwise (e.g., by sending any suitable data 99 for adjusting the light intensity and/or chromaticity and/or temperature and/or sound level of light and/or sound emitted from an auxiliary environment subsystem 200 to improve the comfort level of the user (e.g., to reduce blue light and turn on soothing white noise to increase the user's comfort level for sleep (e.g., when a determined planned or useful user activity is sleep (e.g., when it has been determined a user has not slept recently and just returned home from a cross-time zone business trip)))), and/or for controlling any suitable functionality of any suitable sensor assembly of device 100 or otherwise (e.g., for turning on or off a particular type of sensor and/or for adjusting the functionality (e.g., the accuracy) of a particular type of sensor (e.g., to gather any additional suitable sensor data)), and/or for updating or supplementing any input data available to activity application 103a that may be used to determine a planned activity, and/or the like. For example, a particular condition may be a minimum threshold score below which the predicted comfort score ought to result in a warning or other suitable instruction being provided to the experiencing entity with respect to the unsuitability of the environment of interest with respect to the experiencing entity's comfort (e.g., an instruction to leave or not visit the environment of interest). A threshold score may be determined in any suitable manner and may vary between different experiencing entities and/or between different environments of interest and/or between different combinations of such experiencing entities and environments and/or in any other suitable manner.

It is to be understood that a user (e.g., experiencing entity) does not have to be physically present (e.g., with user device 100) at a particular environment of interest in order for the comfort model to provide a comfort score (e.g., comfort state data) applicable to that environment for that user. Instead, for example, the user may select a particular environment of interest from a list of possible environments of interest (e.g., environments previously experienced by the user or otherwise accessible by the model custodian) as well as any suitable time (e.g., time period in the future or the current moment in time) and/or any suitable planned activity for the environment of interest, and the model custodian may be configured to access any suitable environment category data for that environment of interest (e.g., using any suitable auxiliary environment subsystem data 91 from any suitable auxiliary environment subsystem 200 associated with the environment of interest) in order to determine an appropriate comfort score for that environment of interest and/or to generate any suitable control data for that comfort score, which may help the user determine whether or not to visit that environment.

If an environment of interest is experienced by the experiencing entity, then any suitable environmental behavior data (e.g., any suitable user behavior information), which may include an experiencing entity provided comfort score, may be detected during that experience and may be stored (e.g., along with any suitable environmental characteristic information of that experience) as environmental behavior data 105b and/or may be used in an additional receipt and train loop for further training the learning engine. Moreover, in some embodiments, a comfort model custodian may be operative to compare a predicted comfort score for a particular environment of interest with an actual experiencing entity provided comfort score for the particular environment of interest that may be received after or while the experiencing entity may be actually experiencing the environment of interest and enabled to actually score the environment of interest (e.g., using any suitable user behavior information, which may or may not include an actual user provided score feedback). Such a comparison may be used in any suitable manner to further train the learning engine and/or to specifically update certain features (e.g., weights) of the learning engine. For example, any algorithm or portion thereof that may be utilized to determine a comfort score may be adjusted based on the comparison. A user (e.g., experiencing entity (e.g., an end user of device 100)) may be enabled by the comfort model custodian to adjust one or more filters, such as a profile of environments they prefer and/or any other suitable preferences or user profile characteristics (e.g., age, weight, hearing ability, etc.) in order to achieve such results. This capability may be useful based on changes in an experiencing entity's capabilities and/or objectives as well as the comfort score results. For example, if a user loses its hearing or ability to see color, this information may be provided to the model custodian, whereby one or more weights of the model may be adjusted such that the model may provide appropriate scores in the future.

Therefore, any suitable comfort model custodian (e.g., device 100 and/or auxiliary comfort subsystem 250) may be operative to generate and/or manage any suitable comfort model or comfort learning engine that may utilize any suitable machine learning, such as one or more artificial neural networks, to analyze certain environment data of an environment to predict/estimate the comfort score or comfortness of that environment for a particular user (e.g., generally, and/or at a particular time, and/or with respect to one or more planned activities), which may enable intelligent suggestions be provided to the user and/or intelligent system functionality adjustments be made for improving the user's experiences. For example, a comfort engine may be initially configured or otherwise developed for an experiencing entity based on information provided to a model custodian by the experiencing entity that may be indicative of the experiencing entity's specific preferences for different environments and/or environment types (e.g., generally and/or for particular times and/or for particular planned activities) and/or of the experiencing entity's specific experience with one or more specific environments. An initial version of the comfort engine for the experiencing entity may be generated by the model custodian based on certain assumptions made by the model custodian, perhaps in combination with some limited experiencing entity-specific information that may be acquired by the model custodian from the experiencing entity prior to using the comfort engine, such as the experiencing entity's preference for warm temperatures when sleeping and preference for cold temperatures when exercising. The initial configuration of the comfort engine may be based on data for several environment categories, each of which may include one or more specific environment category data values, each of which may have any suitable initial weight associated therewith, based on the information available to the model custodian at the time of initial configuration of the engine (e.g., at operation 402 of process 400 of FIG. 4). As an example, an environment category may be temperature, and the various specific environment category data values for that environment category may include <0° Celsius, 0-19° Celsius, 20-39° Celsius, 40-59° Celsius, 60-790 Celsius, 80-990 Celsius, and ≥100° Celsius. As another example, an environment category may location type, and the various specific environment category data values for that environment category may include library, park, gym, bedroom or hotel room, and classroom, each of which may have a particular initial weight associated with it. As yet another example, an environment category may be white point chromaticity, and the various specific environment category data values for that environment category may include [0, 0], [¼, ¼], and [½, ½], each of which may have a particular initial weight associated with it.

Once an initial comfort engine has been created for an experiencing entity, the model custodian may provide a survey to the experiencing entity that asks for specific information with respect to a particular environment that the experiencing entity has experienced in the past or which the experiencing entity is currently experiencing. Not only may a survey ask for objective information about a particular environment, such as an identification of the environment, the time at which the environment was experienced, the current sleep level of the experiencing entity, the current nutrition level of the experiencing entity, the current mindfulness level of the experiencing entity, an activity performed by the experiencing entity in the environment, and/or the like, but also for subjective information about the environment, such as the experiencing entity's comfort level in the environment generally or with respect to different environment characteristics (e.g., the experiencing entity's comfort level with respect to the environment's temperature, the experiencing entity's comfort level with respect to the environment's noise level, the experiencing entity's comfort level with respect to the environment's white point chromaticity, the experiencing entity's comfort level with respect to the environment's humidity, etc.) and/or the like. A completed survey may include responses to one or more of the questions as well as an overall score for the environment (e.g., on a scale of 1-10 with 1 being indicative of an environment that was not comfortable to the experiencing entity and with a 10 being indicative of an environment that was extremely comfortable for the experiencing entity, with such success being gauged using any suitable criteria as may be suggested by the model custodian and/or as may be determined by the experiencing entity itself). Each completed experiencing entity survey for one or more environments (e.g., one or more environments generally and/or for one or more times and/or for one or more planned activities) by one or more particular experiencing entity respondents of the experiencing entity may then be received by the model custodian and used to train the comfort engine. By training the comfort engine with such experiencing entity feedback on one or more prior and/or current environment experiences, the comfort engine may be more customized to the likes and dislikes of the experiencing entity by adjusting the weights of one or more environment category options to an updated set of weights for providing an updated comfort engine.

Such an updated comfort engine, as trained based on experiencing entity survey responses or otherwise, may then be used by the model custodian to identify one or more environments that may provide a comfortable experience to an experiencing entity. For example, environment data from each one of one or more available environments accessible to the system (e.g., to the model custodian), for example, in any suitable environment database that may be accessible in any suitable manner (e.g., by the comfort model) may be run through the updated comfort engine for the experiencing entity so as to generate a predicted score for each available environment (e.g., a score between 1-10 that the engine predicts the experiencing entity would rate the available environment if the experiencing entity were to experience in the available environment). If a predicted score is generated by an experiencing entity's comfort engine for a particular available environment that meets a particular threshold (e.g., a score above a 7.5) (e.g., generally or for particular time and/or for a particular planned activity that may be determined to be of interest to the experiencing entity, for example, with respect to an environment that may be within any suitable distance of the current location of the experiencing entity such that it may be practically accessed by the experiencing entity), then the model custodian may utilize that information in any suitable way to facilitate suggesting or otherwise leading the experiencing entity to the particular available environment. Therefore, a model custodian may be used to determine a comfortness match between a user and a particular available environment and to facilitate utilization of a such a determined match. If a user and an environment are matched, any suitable feedback (e.g., environmental behavior data (e.g., environmental characteristic information, user behavior information, user environmental preference(s), and/or the like)) may be obtained by the model custodian (e.g., while the user prepares to experience the environment, during the user's experience of the environment, and/or after the user's experience of the environment) to bolster any suitable environment data associated with that experience in any suitable experience database that may be associated with the model (e.g., in any suitable environment database) and/or to further train the comfort model. Therefore, the comfort engine may be continuously refined and updated by taking into account all feedback provided by any experiencing entity, such that the experiencing entity's comfort engine may be improved for generating more accurate predicted scores going forward for future potential environment experiences. A model custodian may manage not only an environment database and one or more various comfort models (e.g., for one or more different experiencing entities), but also any and/or all connections and/or experiences between experiencing entities and environments, such that the model custodian may be a master interface for all the needs of any experiencing entity and/or of any environment custodian (e.g., a manager of a school or of a park or the like that may benefit from any data that such a model custodian may be able to provide such an environment custodian (e.g., to improve the quality and/or popularity of the environment)).

It is to be understood that device 100 may be a model custodian for at least a portion or all of model 105a and/or for at least a portion or all of model 255a at the same time and/or at different times, and/or subsystem 250 may be a model custodian for at least a portion or all of model 105a and/or for at least a portion or all of model 255a at the same time and/or at different times. Model 105a may be for one or more particular users (e.g., one or more particular users associated with (e.g., registered to) device 100) while model 255a may be for a larger group of experiencing entities, including those of model 105a as well as other users (e.g., users of various other user electronic devices that may be within system 1 (not shown (e.g., within a user device ecosystem)). At least a portion of model 255a may be used with at least a portion of model 105a (e.g., as a hybrid model) in any suitable combination for any suitable purpose, or model 255a may be periodically updated with any suitable model data from model 105a or vice versa. Alternatively, model 105a and model 255a may be identical and only one may be used (e.g., by device 100) for a particular use case.

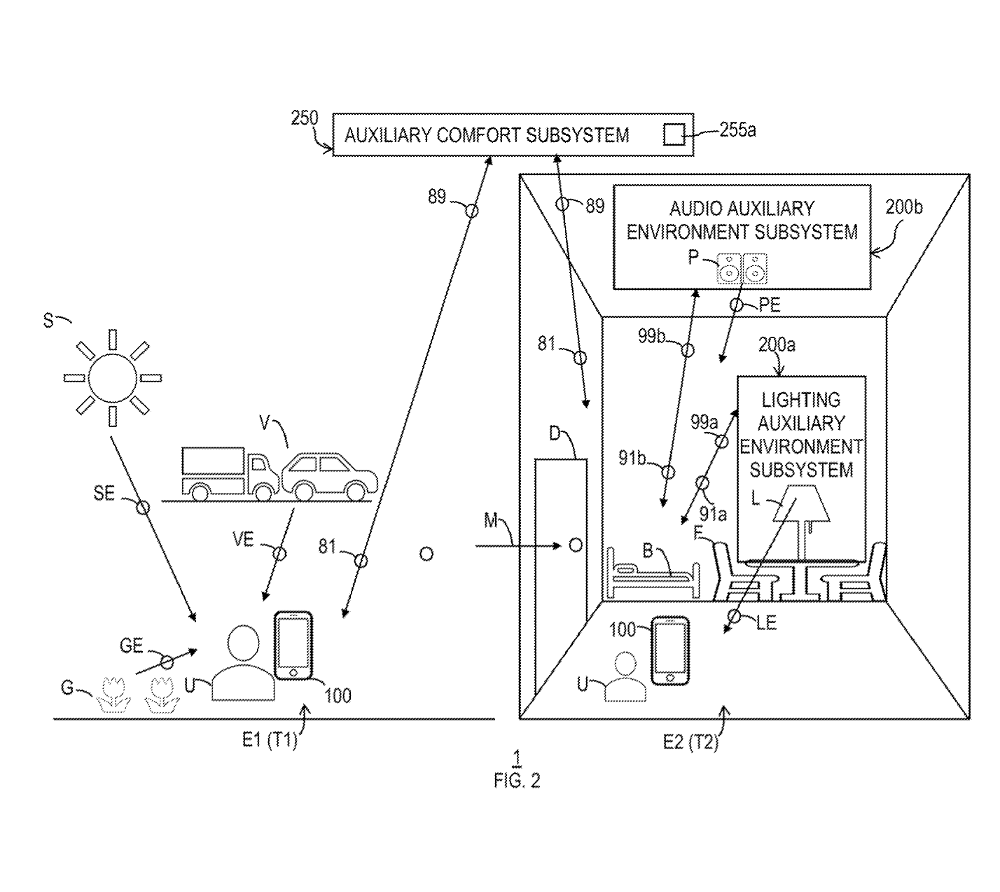

FIG. 2 shows system 1 implemented amongst various environments within which device 100 may be located, such as a first environment E1 (e.g., at a first time T1) and a second environment E2 (e.g., at a second time T2). As shown, as just one specific example, electronic device 100 may be a handheld or otherwise portable electronic device, such as an iPhone™, that may be carried by or otherwise brought with a user U wherever it travels, such as in the direction of arrow M through a door D that may provide a passageway for user U and device 100 between environment E1 and environment E2. As shown, environment E1 may be an outdoor environment that may include a sun S, one or more vehicles V, and a garden G, at least during a first time period T1 during which user U and device 100 may be present at environment E1. Additionally, as shown, environment E2 may be an indoor environment that may include door D, a bed B, any suitable furniture F on which a lamp assembly L of lighting auxiliary environment subsystem 200a may be positioned, and an audio speaker assembly P of an audio auxiliary environment subsystem 200b, at least during a second time period T2 during which user U and device 100 may be present at environment E2. As also shown, auxiliary comfort subsystem 250 may also be accessible to device 100 at each one of environments E1 and E2. However, it is to be understood that environments E1 and E2 of FIG. 2 are only illustrative and that any suitable environments, such as any environment with or without any suitable type(s) of auxiliary environment subsystem(s) 200 (e.g., any suitable appliance and/or controllable device or subsystem in an environment), and/or with or without any suitable features, and/or with or without access to any suitable auxiliary comfort subsystem 250, may be environments in which user U may use device 100 (e.g., a smart home, smart office, smart car, human-centered (“HC”) building management system or building control system (e.g., a building may be configured to communicate with a user's device and adjust the environment to the user's preferred conditions)). Although FIG. 2 may show user U traveling with a portable device 100 between environments, it is to be understood that system 1 need not rely on any portable devices or subsystems. Instead, for example, different environments may include different devices 100 and/or different subsystems 200 and/or different subsystems 250, one or more of which may be operative to detect user U and/or determine one or more appropriate activities of the user and/or one or more environmental characteristics in order to determine appropriate comfort state data 322 and/or to determine appropriate comfort mode data 324 for facilitating features of this disclosure.

At each environment, any or all environmental characteristic information may be sensed by device 100 from any or all features of the environment (e.g., directly via sensor assembly 114 of device 100 and/or via any suitable auxiliary environment subsystem(s) 200 of the environment). For example, as shown, at environment E1 during time T1, sun S may provide one or more types of sun effects SE that may be sensed by sensor assembly 114 of device 100 for determining one or more environmental characteristics of environment E1 during time T1, including, but not limited to, a temperature environmental characteristic of environment E1 that may be at least partially detected from a sensed heat sun effect SE generated by sun S, an illuminance light level environmental characteristic of environment E1 that may be at least partially detected from a sensed light sun effect SE generated by sun S, an ambient color or true color or white point chromaticity environmental characteristic of environment E1 that may be at least partially detected from a color sun effect SE generated by sun S, a UV index environmental characteristic of environment E1 that may be at least partially detected from a UV sun effect SE generated by sun S, and/or the like. As another example, as shown, at environment E1 during time T1, vehicle(s) V may provide one or more types of vehicle effects VE that may be sensed by sensor assembly 114 of device 100 for determining one or more environmental characteristics of environment E1 during time T1, including, but not limited to, a noise environmental characteristic of environment E1 that may be at least partially detected from a sensed noise vehicle effect VE generated by vehicle(s) V, a harmful gas level environmental characteristic of environment E1 that may be at least partially detected from a sensed gas vehicle effect VE generated by vehicle(s) V, and/or the like. As yet another example, as shown, at environment E1 during time T1, garden G may provide one or more types of garden effects GE that may be sensed by sensor assembly 114 of device 100 for determining one or more environmental characteristics of environment E1 during time T1, including, but not limited to, an oxygen level environmental characteristic of environment E1 that may be at least partially detected from a sensed oxygen level garden effect GE generated by garden G, a particulate gas level environmental characteristic of environment E1 that may be at least partially detected from a particulate garden effect GE generated by garden G, and/or the like. Auxiliary comfort subsystem data 81 (e.g., a portion or the entirety of model 255a) may also be detected or otherwise received by device 100 from auxiliary comfort subsystem 250 at environment E1 during time T1 (e.g., automatically and/or in response to any suitable request auxiliary comfort subsystem data 89 that may be communicated to auxiliary comfort subsystem 250). Moreover, as shown, at environment E2 during time T2, lamp L may provide one or more types of lamp effects LE that may be sensed by sensor assembly 114 of device 100 for determining one or more environmental characteristics of environment E2 during time T2, including, but not limited to, a temperature environmental characteristic of environment E2 that may be at least partially detected from a sensed heat lamp effect LE generated by lamp L, an illuminance light level environmental characteristic of environment E2 that may be at least partially detected from a sensed light lamp effect LE generated by lamp L, an ambient color or true color or white point chromaticity environmental characteristic of environment E2 that may be at least partially detected from a color lamp effect LE generated by lamp L, a UV index environmental characteristic of environment E2 that may be at least partially detected from a UV lamp effect LE generated by lamp L, and/or the like. Additionally or alternatively, any suitable auxiliary environment subsystem data 91a may be communicated to device 100 from lighting auxiliary environment subsystem 200a (e.g., automatically and/or in response to any suitable request auxiliary environment subsystem data 99a that may be communicated to lighting auxiliary environment subsystem 200a) that may be indicative of any suitable sensed lamp effect and/or any suitable output characteristic of any suitable output assembly (e.g., lamp output assembly L) of subsystem 200a and/or the like that may be available to subsystem 200a. As another example, as shown, at environment E2 during time T2, speaker(s) P may provide one or more types of speaker effects PE that may be sensed by sensor assembly 114 of device 100 for determining one or more environmental characteristics of environment E2 during time T2, including, but not limited to, a noise environmental characteristic of environment E2 that may be at least partially detected from a sensed noise speaker effect PE generated by speaker(s) P, and/or the like. Additionally or alternatively, any suitable auxiliary environment subsystem data 91b may be communicated to device 100 from audio auxiliary environment subsystem 200b (e.g., automatically and/or in response to any suitable request auxiliary environment subsystem data 99b that may be communicated to audio auxiliary environment subsystem 200b) that may be indicative of any suitable sensed speaker effect and/or any suitable output characteristic of any suitable output assembly (e.g., speaker output assembly P) of subsystem 200b and/or the like that may be available to subsystem 200b. Auxiliary comfort subsystem data 81 (e.g., a portion or the entirety of model 255a) may also be detected or otherwise received by device 100 from auxiliary comfort subsystem 250 at environment E2 during time T2. Any other suitable environmental characteristic information may be detected by device 100 or otherwise by system 1 for a particular environment at a particular time in any suitable manner (e.g., by a model custodian or otherwise, whether or not device 100 may be present at that environment or not), such as physical location environmental characteristic information (e.g., geo-location, address, location type (e.g., zoo, home, office, school, park, etc.) using any suitable data (e.g., via GPS data)), time zone environmental characteristic information, humidity characteristic information, and/or the like.

Such environmental characteristic information, which may be sensed or otherwise received by device 100 or any other suitable subsystem of system 1 (e.g., any suitable model custodian) for a particular environment at a particular time, may be processed and/or stored by that subsystem as at least a portion of environmental behavior data 105b alone or in conjunction with any suitable user behavior information that may be provided by user U (e.g., by input assembly 110) or otherwise detected by device 100 (e.g., by sensor assembly 114) that may be indicative of a user's behavior within and/or reaction to the particular environment, for example, as at least another portion of environmental behavior data 105b. Any suitable user behavior information for a user at a particular environment at a particular time may be detected in any suitable manner by device 100 or any other suitable subsystem. For example, any specific user-provided feedback information may be provided by user U to device 100 (e.g., via any suitable input assembly 110 (e.g., typed via a keyboard or dictated via a user microphone, etc.)) or to any suitable subsystem 200 (e.g., by an input assembly of a subsystem 200b (e.g., a user turning the volume of speaker P of subsystem 200b up via an input assembly of subsystem 200b)) that may then be shared with device 100 (e.g., as data 91)) that may be indicative of the user's comfort level in the particular environment at the particular time (e.g., a subjective user-provided ranking (e.g., on a scale of 1-10), generally or for a particular activity (e.g., for exercising, for sleeping, for studying, etc.), and/or a subjective user-provided preference for adjusting the environment in some way (e.g., too hot, too loud, etc.), generally or for a particular activity (e.g., for exercising, for sleeping, for studying, etc.)). Such user-provided feedback may be requested by device 100 to the user via any suitable user interface application and/or via any suitable output assembly 112 (e.g., via a display output assembly or via an audio speaker output assembly based on a device user interface application). As another example, user activity behavior information indicative of a behavior of user U may be detected by sensor assembly 114 of device 100 that may be indicative of the user's performance of any suitable activity in the particular environment at the particular time (e.g., any suitable exercise activity information, any suitable sleep information, any suitable mindfulness information, etc.), which may be indicative of the user's effectiveness or ability to perform an activity within the particular environment. Such environmental characteristic information that may be sensed or otherwise received by device 100 for a particular environment at a particular time, as well as such user behavior information that may be sensed or otherwise received by device 100 for the particular environment at the particular time, may together be processed and/or stored by device 100 as at least a portion of environmental behavior data 105b (e.g., for tracking a user's subjective comfort level for a particular environment at a particular time and/or a user's objective activity performance capability for a particular environment at a particular time). For example such environmental behavior data 105b may be used as at least a portion of any suitable environment data that may be used by a comfort model to determine a comfort score for that environment for that user and/or to train such a comfort model in order to better prepare that comfort model for a future comfort score determination.

To accurately predict the comfort that may be provided by an environment to a user, any suitable portion of system 1, such as device 100, may be configured to use various information sources in combination with any available comfort model in order to characterize or classify or predict a comfort level or a comfort state of a user of device 100 when appropriate or when possible. For example, any suitable processing circuitry or assembly (e.g., a comfort module) of device 100 may be configured to gather and to process various types of environment data, in conjunction with a comfort model, to determine what type of comfort level is to be expected for a particular environment. For example, any suitable environment data from one or more of sensor assembly 114 of device 100, auxiliary environment subsystem 200 (e.g., from one or more assemblies thereof), activity application 103a of device 100, and/or environmental behavior data 105b of device 100 may be utilized in conjunction with any suitable comfort model, such as with device comfort model 105a and/or auxiliary comfort model 255a, to determine a comfort state of a user efficiently and/or effectively.

FIG. 3 shows a schematic view of a comfort management system 301 of electronic device 100 that may be provided to manage comfort states of device 100 (e.g., to determine a comfort state of device 100 and to manage a mode of operation of device 100 and/or of any other suitable subsystem of system 1 based on the determined comfort state). In addition to or as an alternative to using device sensor assembly data 114′ that may be generated by device sensor assembly 114 based on any sensed environment characteristics, comfort management system 301 may use various other types of data accessible to device 100 in order to determine a current comfort state of a user of device 100 in a particular environment and/or to determine a predicted comfort state of a user in an available environment in conjunction with any suitable comfort model (e.g., in conjunction with model 105a and/or model 255a), such as any suitable data provided by one or more of auxiliary environment subsystem 200 (e.g., data 91 from one or more assemblies of auxiliary environment subsystem 200), activity application 103a of device 100 (e.g., data 103a′ that may be provided by application 103a and that may be indicative of one or more planned activities), and/or environmental behavior data 105b (e.g., any suitable environmental behavior data 105b′ that may be any suitable portion or the entirety of environmental behavior data 105b). In response to determining the current comfort state for a current environment or a predicted comfort state for a potential available environment, comfort management system 301 may apply at least one comfort-based mode of operation to at least one managed element 390 (e.g., any suitable assembly of device 100 and/or any suitable assembly of subsystem 200 and/or any suitable assembly of subsystem 250 or otherwise of system 1) based on the determined comfort state (e.g., to suggest certain user behavior and/or to control the functionality of one or more system assemblies) for improving a user's experience. For example, as shown in FIG. 3, comfort management system 301 may include a comfort module 340 and a management module 380.

Comfort module 340 of comfort management system 301 may be configured to use various types of data accessible to device 100 in order to determine (e.g., characterize) a comfort state (e.g., a current comfort state of a user of device 100 within a current environment and/or a potential comfort state of a user within a potential available environment). As shown, comfort module 340 may be configured to receive any suitable device sensor assembly data 114′ that may be generated and shared by any suitable device sensor assembly 114 based on any sensed environment characteristics (e.g., automatically or in response to any suitable request type of device sensor request data 114″ that may be provided to sensor assembly 114), any suitable auxiliary environment subsystem data 91 that may be generated and shared by any suitable auxiliary environment subsystem assembly(ies) based on any sensed environmental characteristics or any suitable auxiliary subsystem assembly characteristics (e.g., automatically or in response to any suitable request type of auxiliary environment subsystem data 99′ that may be provided to auxiliary environment subsystem 200), any suitable activity application status data 103a′ that may be generated and shared by any suitable activity application 103a that may be indicative of one or more planned activities (e.g., automatically or in response to any suitable request type of activity application request data 103a″ that may be provided to activity application 103a), and/or any suitable environmental behavior data 105b′ that may be any suitable shared portion or the entirety of environmental behavior data 105b (e.g., automatically or in response to any suitable request type of environmental behavior request data 105b″ that may be provided to a provider of environmental behavior data 105b (e.g., memory assembly 104), and comfort module 340 may be operative to use such received data in any suitable manner in conjunction with any suitable comfort model to determine any suitable comfort state (e.g., with device comfort model data 105a′ that may be any suitable portion or the entirety of device comfort model 105a, which may be accessed automatically and/or in response to any suitable request type of device comfort model request data 105a″ that may be provided to a provider of device comfort model 105a (e.g., memory assembly 104), and/or with auxiliary comfort subsystem model data 81 that may be any suitable portion or the entirety of auxiliary comfort model 255a, which may be accessed automatically and/or in response to any suitable request type of auxiliary comfort subsystem request data 89′ that may be provided to a provider of auxiliary comfort model 255a (e.g., auxiliary comfort subsystem 250)).

Once comfort module 340 has determined a current comfort state for a current environment or a predicted comfort state for a potential available environment (e.g., based on any suitable combination of one or more of any suitable received data 114′, 91, 103a′, 105b′, 105a′, and 81), comfort module 340 may be configured to generate and transmit comfort state data 322 to management module 380, where comfort state data 322 may be indicative of the determined comfort state for the user of device 100. In response to determining a comfort state of a user of device 100 by receiving comfort state data 322, management module 380 may be configured to apply at least one comfort-based mode of operation to at least one managed element 390 of device 100 based on the determined comfort state. For example, as shown in FIG. 3, comfort management system 301 may include management module 380, which may be configured to receive comfort state data 322 from comfort module 340, as well as to generate and share comfort mode data 324 with at least one managed element 390 of device 100 and/or of any other suitable subsystem of system 1 at least partially based on the received comfort state data 322, where such comfort mode data 324 may be received by managed element 390 for controlling at least one characteristic of managed element 390. Managed element 390 may be any suitable assembly of device 100 (e.g., any processor assembly 102, any memory assembly 104 and/or any data stored thereon, any communications assembly 106, any power supply assembly 108, any input assembly 110, any output assembly 112, any sensor assembly 114, etc.) and/or any suitable assembly of any suitable auxiliary environment subsystem 200 of system 1 and/or any suitable assembly of any suitable auxiliary comfort subsystem 250 of system 1, and comfort mode data 324 may control managed element 390 in any suitable way, such as by enhancing, enabling, disabling, restricting, and/or limiting one or more certain functionalities associated with such a managed element.

Comfort mode data 324 may be any suitable device control data for controlling any suitable functionality of any suitable assembly of device 100 as a managed element 390 (e.g., any suitable device output control data for controlling any suitable functionality of any suitable output assembly 112 of device 100 (e.g., for adjusting a user interface presentation to user U (e.g., to provide a comfort suggestion or a comfort score)), and/or any suitable device sensor control data (e.g., a control type of device sensor request data 114″) for controlling any suitable functionality of any suitable sensor assembly 114 of device 100 (e.g., for turning on or off a particular type of sensor and/or for adjusting the functionality (e.g., the accuracy) of a particular type of sensor (e.g., to gather any additional suitable sensor data)), and/or any suitable activity application control data (e.g., a control type of activity application request data 103a″) for updating or supplementing any input data available to activity application 103a that may be used to determine a planned activity, and/or the like). Additionally or alternatively, comfort mode data 324 may be any suitable auxiliary environment subsystem data 99 for controlling any suitable functionality of any suitable auxiliary environment subsystem 200 as a managed element 390 (e.g., any suitable auxiliary environment subsystem data 99a for controlling any suitable functionality of lighting auxiliary environment subsystem 200a (e.g., for adjusting a lighting characteristic of lamp L, etc.), any suitable auxiliary environment subsystem data 99b for controlling any suitable functionality of audio auxiliary environment subsystem 200b (e.g., for adjusting a sound characteristic of speaker P, etc.), and/or the like). Additionally or alternatively, comfort mode data 324 may be any suitable auxiliary comfort subsystem data 89 for providing any suitable data to auxiliary comfort subsystem 250 as a managed element 290 (e.g., any suitable auxiliary comfort subsystem data 89 for updating auxiliary comfort model 255a of auxiliary comfort subsystem 250 in any suitable manner). Additionally or alternatively, comfort mode data 324 may be any suitable device comfort model update data (e.g., an update type of device comfort model request data 105a″) for providing any suitable data to device comfort model 105a as a managed element 390 (e.g., any suitable device comfort model update data 105a″ for updating device comfort model 105a in any suitable manner). Additionally or alternatively, comfort mode data 324 may be any suitable device environmental behavior update data (e.g., an update type of environmental behavior request data 105b″) for providing any suitable update data to environmental behavior data 105b as a managed element 390 (e.g., any suitable environmental behavior update data 105b″ for updating environmental behavior data 105b in any suitable manner).

FIG. 4 is a flowchart of an illustrative process 400 for managing a comfort level. At operation 402 of process 400, a comfort model custodian (e.g., a comfort model custodian system) may initially configure a learning engine (e.g., device comfort model 105a) for an experiencing entity. At operation 404 of process 400, the comfort model custodian may receive, from the experiencing entity, environment category data for at least one environment category for an environment and a score for the environment. At operation 406 of process 400, the comfort model custodian may train the learning engine using the received environment category data and the received score. At operation 408 of process 400, the comfort model custodian may access environment category data for the at least one environment category for another environment. At operation 410 of process 400, the comfort model custodian may score the other environment, using the learning engine, with the accessed environment category data for the other environment. At operation 412 of process 400, when the score for the other environment satisfies a condition, the comfort model custodian may generate control data associated with the satisfied condition.

It is understood that the operations shown in process 400 of FIG. 4 are only illustrative and that existing operations may be modified or omitted, additional operations may be added, and the order of certain operations may be altered.

Therefore, systems and methods may be provided for assessing the subjective comfortness level of an individual based on measurements of physical attributes of an environment. Various sensor assemblies provided by a user electronic device, including white point chromaticity color sensors, temperature sensors, air quality sensors, location sensors, and/or the like, on their own or in combination with any suitable remote auxiliary subsystem assemblies, may be capable of collecting extensive data about a current environment of a device user and/or a potential available environment of a device user. Combined with any suitable psychophysical experimental results and/or individual user preferences and/or behavior, such environmental information elements may be utilized (e.g., using any suitable model or engine or neural network or the like) to evaluate and/or monitor the comfortness or comfortableness of comfort level or comfort state of an environment (e.g., generally or for a particular type or subset of user or for a particular user (e.g., generally or for a particular time and/or for a particular planned activity)). Such a comfort level may be used to generate alerts about hazardous conditions and/or make recommendations or suggestions about environment modifications and/or the like.

Certain regulatory standards or thresholds for certain environmental characteristics for certain types of environments (e.g., a maximum temperature threshold for a school, a minimum illuminance threshold for an office, a maximum harmful gas level for a laboratory, etc.) may be made available to the system for enabling detection of not only a comfort level but also detecting and alerting any hazardous or illegal conditions that may be presented by a particular environment (e.g., generally or for a particular user and/or for a particular time and/or for a particular activity (e.g., too humid to safely exercise, too dark to safely read, etc.)). The system may provide any suitable comfort mode data 324 that may be operative to guide efforts in improving productivity of employees (e.g., making lights brighter, making sound quieter, providing predicted employee comfort levels, etc.). The system may provide any suitable comfort state data 322 that may be indicative of an overall comfort quality metric for a particular environment (e.g., generally or for a particular user and/or for a particular time and/or for a particular activity) and/or that may be indicative of a particular comfort quality metric for a particular environment characteristic of a particular environment (e.g., a comfort level score for light level of an environment or white point chromaticity of an environment or noise level or UV index or humidity or the like (e.g., generally or for a particular user and/or for a particular time and/or for a particular activity)).

Environmental behavior data 105b may be tracked for historical records of any suitable environmental characteristic information and/or of any suitable user activity behavior information, such as a record of the intensity, duration, and/or time occurrence of any suitable external stimuli that may affect user's level of comfortness (e.g., noise level, light level, chromaticity of ambient light and its intensity, temperature, UV index, harmful gas and oxygen concentration in air, etc.). Analysis of such historical data (e.g., historical data of ambient light chromaticity) may be used for any suitable applications (e.g., for any suitable managed element), such as any suitable sleep tracking application (e.g., for monitoring how a user's sleep performance may be related to its exposure to certain color light). Any suitable suggestions may be made to a system user and/or any suitable automatic functionality adjustment of a system assembly may be made based on historical data analysis and/or any suitable comfort level determination, including, but not limited to, adjustment of light level, adjustment of chromaticity of light, adjustment of temperature, adjustment of sound level, adjustment of humidity or suggest to avoid excessive humidity or to move to a less humid environment (e.g., to exercise), suggestion to move to a less noisy environment (e.g., to study or sleep), and/or the like.

The system may be operative to track a historical record of the intensity, duration, and time occurrence of external stimuli, and/or store historical statistics of the comfortableness or satisfaction or conduciveness or usefulness or effectiveness or contribution of one or more various environments (e.g., generally or for a particular user and/or for a particular time and/or for a particular activity). The system may be operative to provide recommendations and alerts when the comfortness fails or exceeds certain thresholds. Based on any suitable environment data, the system may be operative to provide suggestions as to how a user might improve environment conditions in order to increase the level of comfortness, or, for example, to improve sleep quality or reduce the effect of desynchronosis or circadian dysrhythmia (i.e., jet lag). Other physiological information (e.g., number of steps, flights climbed, calories burnt, walking/running distance, sleep quality, mindfulness quality, nutritional quality, alertness quality, etc.) could be combined with or provided as any suitable environmental data in order to train the system to correlate with a psychophysical experiment result (e.g., using any suitable comfort model). Various types of data may be used to train any suitable comfort model, such as any suitable acts or regulations or best practices that may be applicable to one or more environments and/or locations and/or users (e.g., user conditions (e.g., diseases, etc.)) and/or activities, any suitable preference user studies, any suitable recommendations for comfort zones, and/or the like. For example, a wide user study may be conducted for various particular or generic environments in order to obtain data useful for initially training such a model. Based on a user's preferences, a deployed system may be operative to train itself (e.g., to predict a user's comfort level, to provide alerts in accordance with any suitable acts and regulations, recommend modifications of user behavior and/or system assembly functionality, and/or the like). Such a system may identify and provide an improved user experience based on any suitable environment comfortability traits, such as general traits, including, but not limited to, excessive humidity or temperature may deteriorate productivity, clear blue sky with a bright sun may make people happier than an overcast sky on a rainy day, the color of ambient light may affect a person's mood and/or well-being and/or circadian rhythms and/or productivity and/or the like, critical levels of toxic gases or oxygen may have negative health effects, a noisy office may deteriorate productivity, and/or the like.

Various suggestions or messages may be provided to a user in response to various comfort determinations for various environments, such as, “concentrate on breathing for 30 seconds”, “go outside for 2 minutes to feel the sun”, “decrease the temperature of this environment in order to create a more exercise-conducive environment”, “increase the illuminance of this environment in order to create a more study-conducive environment,” “lift weights rather than run in this environment”, “increase temperature by 5° Celsius to align this environment with your ideal sleeping environment (e.g., based on historical data indicative of when you sleep best)”, “wait until humidity decreases by 10% to align your environment with your ideal running environment (e.g., based on historical data indicative of when you run best)”, “this environment is ranked an 8 comfort level for running, a 6 comfort level for sleeping, and a 4 comfort level for studying”, and/or the like. In some embodiments, when a user is detected to have transitioned from one environment to another (e.g., from outdoor environment E1 to indoor environment E2 of FIG. 2), the system may be operative to compute or utilize a moving average or a transition discount or any other suitable technique that may reduce an abrupt effect of such a transition (e.g., if the temperature difference between environment E1 and environment E2 is above a particular threshold (e.g., greater than 50° Celsius), then the system may delay any recommendation to adjust the temperature at the new environment in order to enable the user first to more naturally adjust its comfort level to the new environment.

The use of one or more suitable models or engines or neural networks or the like (e.g., device comfort model 105a) may enable prediction or any suitable determination of an appropriate comfort state of a user at a particular environment. Such models (e.g., neural networks) running on any suitable processing units (e.g., graphical processing units (“GPUs”) that may be available to system 1) provide significant speed improvements in efficiency and accuracy with respect to prediction over other types of algorithms and human-conducted analysis of data, as such models can provide estimates in a few milliseconds or less, thereby improving the functionality of any computing device on which they may be run. Due to such efficiency and accuracy, such models enable a technical solution for enabling the generation of any suitable control data (e.g., for controlling any suitable functionality of any suitable output assembly of an electronic device or of any subsystem associated with an environment (e.g., for adjusting a user interface presentation to a user (e.g., to provide a comfort suggestion or a comfort score) and/or for adjusting an output that may affect the comfort of the user within the environment (e.g., for adjusting the light intensity, chromaticity, temperature, sound level, etc. of the environment))) using any suitable real-time data (e.g., data made available to the models) that may not be possible without the use of such models, as such models may increase performance of their computing device(s) by requiring less memory, providing faster response times, and/or increased accuracy and/or reliability. Due to the condensed time frame and/or the time within which a decision with respect to environment data ought to be made to provide a desirable user experience, such models offer the unique ability to provide accurate determinations with the speed necessary to enable user comfort.

Moreover, one, some, or all of the processes described with respect to FIGS. 1-4 may each be implemented by software, but may also be implemented in hardware, firmware, or any combination of software, hardware, and firmware. They each may also be embodied as machine- or computer-readable code recorded on a machine- or computer-readable medium. The computer-readable medium may be any data storage device that can store data or instructions which can thereafter be read by a computer system. Examples of such a non-transitory computer-readable medium (e.g., memory assembly 104 of FIG. 1) may include, but are not limited to, read-only memory, random-access memory, flash memory, CD-ROMs, DVDs, magnetic tape, removable memory cards, optical data storage devices, and the like. The computer-readable medium can also be distributed over network-coupled computer systems so that the computer-readable code is stored and executed in a distributed fashion. For example, the computer-readable medium may be communicated from one electronic device to another electronic device using any suitable communications protocol (e.g., the computer-readable medium may be communicated to electronic device 100 via any suitable communications assembly 106 (e.g., as at least a portion of application 103)). Such a transitory computer-readable medium may embody computer-readable code, instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal.

It is to be understood that any or each module of comfort management system 301 may be provided as a software construct, firmware construct, one or more hardware components, or a combination thereof. For example, any or each module of comfort management system 301 may be described in the general context of computer-executable instructions, such as program modules, that may be executed by one or more computers or other devices. Generally, a program module may include one or more routines, programs, objects, components, and/or data structures that may perform one or more particular tasks or that may implement one or more particular abstract data types. It is also to be understood that the number, configuration, functionality, and interconnection of the modules of comfort management system 301 are only illustrative, and that the number, configuration, functionality, and interconnection of existing modules may be modified or omitted, additional modules may be added, and the interconnection of certain modules may be altered.

At least a portion of one or more of the modules of comfort management system 301 may be stored in or otherwise accessible to device 100 in any suitable manner (e.g., in memory assembly 104 of device 100 (e.g., as at least a portion of application 103)). Any or each module of comfort management system 301 may be implemented using any suitable technologies (e.g., as one or more integrated circuit devices), and different modules may or may not be identical in structure, capabilities, and operation. Any or all of the modules or other components of comfort management system 301 may be mounted on an expansion card, mounted directly on a system motherboard, or integrated into a system chipset component (e.g., into a “north bridge” chip).

Any or each module of comfort management system 301 may be a dedicated system implemented using one or more expansion cards adapted for various bus standards. For example, all of the modules may be mounted on different interconnected expansion cards or all of the modules may be mounted on one expansion card. With respect to comfort management system 301, by way of example only, the modules of comfort management system 301 may interface with a motherboard or processor assembly 102 of device 100 through an expansion slot (e.g., a peripheral component interconnect (“PCI”) slot or a PCI express slot). Alternatively, comfort management system 301 need not be removable but may include one or more dedicated modules that may include memory (e.g., RAM) dedicated to the utilization of the module. In other embodiments, comfort management system 301 may be at least partially integrated into device 100. For example, a module of comfort management system 301 may utilize a portion of device memory assembly 104 of device 100. Any or each module of comfort management system 301 may include its own processing circuitry and/or memory. Alternatively, any or each module of comfort management system 301 may share processing circuitry and/or memory with any other module of comfort management system 301 and/or processor assembly 102 and/or memory assembly 104 of device 100.

As described above, one aspect of the present technology is the gathering and use of data available from various sources to improve the determination of comfort states of a user (e.g., a user of an electronic device). The present disclosure contemplates that in some instances, this gathered data may include personal information data that uniquely identifies or can be used to contact or locate a specific person. Such personal information data can include demographic data, location-based data, telephone numbers, email addresses, social network identifiers, home addresses, office addresses, data or records relating to a user's health or level of fitness (e.g., vital signs measurements, medication information, exercise information, etc.) and/or mindfulness, date of birth, or any other identifying or personal information.

The present disclosure recognizes that the use of such personal information data, in the present technology, can be used to the benefit of users. For example, the personal information data can be used to improve the determination of comfort states of a user. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of location detection services, the present technology can be configured to allow users to select to “opt in” or “opt out” of participation in the collection of personal information data during registration for services or anytime thereafter. In addition to providing “opt in” or “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, the determination of comfort states of a user of an electronic device can be made based on non-personal information data or a bare minimum amount of personal information, such as the content being requested by the device associated with a user, other non-personal information available to the device, or publicly available information.

While there have been described systems, methods, and computer-readable media for managing comfort states of a user of an electronic device, it is to be understood that many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

Therefore, those skilled in the art will appreciate that the invention can be practiced by other than the described embodiments, which are presented for purposes of illustration rather than of limitation.

What is claimed is:

1. A method for managing a comfort level of an experiencing entity using a comfort model custodian system, the method comprising:
initially configuring, at the comfort model custodian system, a learning engine for the experiencing entity;
receiving, at the comfort model custodian system from the experiencing entity, environment category data for at least one environment category for an environment and a score for the environment;
training, at the comfort model custodian system, the learning engine using the received environment category data and the received score;
accessing, at the comfort model custodian system, environment category data for the at least one environment category for another environment;
scoring the other environment, using the learning engine for the experiencing entity at the comfort model custodian system, with the accessed environment category data for the other environment; and
when the score for the other environment satisfies a condition, generating, with the comfort model custodian system, control data associated with the satisfied condition.

2. The method of claim 1, wherein the at least one environment category comprises ambient light color.

3. The method of claim 1, wherein the at least one environment category comprises light color and light illuminance.

4. The method of claim 1, wherein the control data is operative to provide a recommendation to adjust the ambient light color of the other environment.

5. The method of claim 1, wherein the control data is operative to automatically adjust an ambient light color of the other environment.

6. The method of claim 1, wherein the at least one environment category comprises a category of environmental characteristic information.

7. The method of claim 6, wherein the category of environmental characteristic information comprises one of the following:
temperature;
noise level;
oxygen level;
air velocity;
humidity;
level of a gas;
geo-location;
location type;
time of day;
day of week;
week of month;
week of year;
month of year;
season;
holiday; or
time zone.

8. The method of claim 1, wherein the at least one environment category comprises a category of user behavior information.

9. The method of claim 8, wherein the category of user behavior information comprises user-provided feedback information provided by a user via an input assembly of a user electronic device.

10. The method of claim 1, wherein the at least one environment category comprises a category of user environmental preferences.

11. The method of claim 10, wherein the category of user environmental preferences comprises one of the following:
a preferred temperature of a user;
a preferred noise level of a user;
a preferred oxygen level of a user;
a preferred air velocity of a user; or
a preferred humidity of a user.

12. The method of claim 1, wherein the at least one environment category comprises a category of planned activity.

13. The method of claim 12, wherein the category of planned activity comprises one of the following:
exercise;
read;
sleep;
study; or
work.

14. The method of claim 1, wherein the control data is operative to provide a recommendation to adjust a temperature of the other environment.

15. The method of claim 1, wherein the control data is operative to automatically adjust a temperature of the other environment.

16. The method of claim 1, wherein the control data is operative to provide a recommendation to adjust a sound level of the other environment.

17. The method of claim 1, wherein the control data is operative to automatically adjust a sound level of the other environment.

18. The method of claim 1, wherein the control data is operative to automatically adjust a functionality of a computing device located at the other environment.

19. A comfort model custodian system comprising:
a communications component; and
a processor operative to:
initially configure a learning engine for an experiencing entity;
receive, from the experiencing entity via the communications component, environment category data for at least one environment category for an environment and a score for the environment;
train the learning engine using the received environment category data and the received score;
access environment category data for the at least one environment category for another environment;
score the other environment, using the learning engine for the experiencing entity, with the accessed environment category data for the other environment; and
when the score for the other environment satisfies a condition, generate control data associated with the satisfied condition.

20. A non-transitory computer-readable storage medium storing at least one program comprising instructions, which, when executed:
initially configure a learning engine for an experiencing entity;
receive, from the experiencing entity, environment category data for at least one environment category for an environment and a score for the environment;
train the learning engine using the received environment category data and the received score;
access environment category data for the at least one environment category for another environment;
score the other environment, using the learning engine for the experiencing entity at the comfort model custodian system, with the accessed environment category data for the other environment; and
when the score for the other environment satisfies a condition, generate control data associated with the satisfied condition.
